(19) United States
(12) Patent Application Publication
(10) Pub. No.: US 2019/0119348 A1
(43) Pub. Date: Apr. 25, 2019

(54) PARATHYROID HORMONE RECEPTOR 1 ANTAGONIST AND INVERSE AGONIST POLYPEPTIDES AND METHODS OF THEIR USE

(71) Applicants: The General Hospital Corporation, Boston, MA (US); Chugai Seiyaku Kabushiki Kaisha, Tokyo (JP)

(72) Inventors: Thomas J. GARDELLA, Needham, MA (US); Tomoyuki WATANABE, Shizuoka (JP)

(73) Assignees: The General Hospital Corporation, Boston, MA (US); Chugai Seiyaku Kabushiki Kaisha, Tokyo (JP)

(21) Appl. No.: 16/090,536

(22) PCT Filed: Mar. 31, 2017
(86) PCT No.: PCT/US2017/025559

§ 371 Date: Oct. 1, 2018

Related U.S. Application Data
(60) Provisional application No. 62/317,152, filed on Apr. 1, 2016.

(51) Int. Cl. C07K 14/635; A61P 5/20

(52) U.S. Cl. CPC C07K 14/635; A61K 38/00; A61P 5/20

(57) ABSTRACT

Parathyroid hormone receptor 1 (PTHR1) antagonist and inverse agonist polypeptides and pharmaceutically acceptable salts thereof are disclosed. The polypeptides include N-terminally truncated PTH/PTHrP hybrid peptides or their fragments. Also disclosed are pharmaceutical compositions containing the PTHR1 antagonists and inverse agonists as well as methods of their use.

### STATEMENT AS TO FEDERALLY FUNDED RESEARCH

This invention was made with Government support under Grant No. NIH-DK-11794 awarded by the National Institutes of Health. The Government has certain rights in the invention.

### FIELD OF THE INVENTION

This invention relates to parathyroid hormone receptor 1 antagonists or inverse agonists. The invention also relates to compositions of the parathyroid hormone peptides and methods of their use.

### BACKGROUND

Excessive signaling activity of parathyroid hormone receptor 1 (PTHR1) is known to be associated with diseases, such as hypercalcemia, hypophosphatemia, hyperparathyroidism, and Jansen's chondrodysplasia. These diseases can arise from overproduction of either of the two endogenous PTHR1 ligands—PTH, as in primary or secondary hyperparathyroidism (HPT), or PTH-related protein (PTHrP), as in humoral hypercalcemia of malignancy. These diseases are characterized by high levels of blood calcium, excessive urinary excretion of calcium and/or phosphate, and can further be associated with abnormal bones, due to alterations in bone formation/resorption activities mediated by the PTHR1.

Regulation of extracellular calcium concentration is necessary for the normal function of the gastrointestinal, skeletal, neurologic, neuromuscular, and cardiovascular systems. PTH synthesis and release are controlled principally by the serum calcium level; a low level stimulates and a high level suppresses both hormone synthesis and release. PTH, in turn, maintains the serum calcium level by directly or indirectly promoting calcium entry into the blood at three sites of calcium exchange: gut, bone, and kidney. PTH contributes to net gastrointestinal absorption of calcium by favoring the renal synthesis of the active form of vitamin D. PTH promotes calcium resorption from bone indirectly by stimulating differentiation of the bone-resorbing cells, osteoclasts. It also mediates at least three main effects on the kidney: stimulation of tubular calcium reabsorption, enhancement of phosphate clearance, and promotion of an increase in the enzyme that completes synthesis of the active form of vitamin D.

Disruption of calcium homeostasis may produce many clinical conditions (e.g., severe bone disease, anemia, renal impairment, ulcers, myopathy, and neuropathy) and usually results from conditions that produce an alteration in the level of parathyroid hormone. Hypercalcemia is a condition that is characterized by an elevation in the serum calcium level. It is often associated with primary hyperparathyroidism in which an excess of PTH production occurs as a result of a parathyroid gland lesion (e.g., adenoma, hyperplasia, or carcinoma). Another type of hypercalcemia, humoral hypercalcemia of malignancy (HHM), is a common paraneoplastic syndrome. It appears to result in most instances from the production by tumors (e.g., squamous, renal, ovarian, or bladder carcinomas) of a class of protein hormone which shares amino acid homology with PTH. These PTH-related proteins (PTHrP) appear to mimic certain of the renal and skeletal actions of PTH and are believed to interact with the PTH receptor in these tissues.

Antagonist ligands for the parathyroid hormone receptor 1 (PTHR1) can be useful for treating diseases associated with excessive signaling activity at the PTHR1. Some of the antagonist ligands may function as inverse agonists.

There is a need for PTHR1 antagonists and inverse agonists, particularly, those that retain high affinity for PTHR1.

### SUMMARY OF THE INVENTION

In general, the present invention provides PTHR1 antagonist or inverse agonist peptides. These peptides can be used in a method of treating a condition or a disease of signaling overactivity of PTHR1. The condition or disease may be associated with higher than normal serum levels of calcium, with lower than normal serum levels of phosphate, with higher than normal levels of endogenous PTHR1 agonist(s), or with constitutive activity of PTHR1 mutants.

In one aspect, the invention provides a polypeptide or a pharmaceutically acceptable salt thereof including an N-terminally truncated PTH/PTHrP hybrid peptide or a fragment thereof (e.g., a fragment containing from 24 to 32 amino acid residues of the N-terminally truncated PTH/PTHrP hybrid peptide), where the polypeptide is a PTHR1 antagonist or a PTHR1 inverse agonist.

In some embodiments, the polypeptide is the N-terminally truncated PTH/PTHrP hybrid peptide is of formula (I):

(I)
Ile-Gln-Leu-X₀₁-His-X₀₂-X₀₃-X₀₄-X₀₅-X₀₆-X₀₇-X₀₈-X₀₉-
X₁₀-Arg-Arg-Arg-X₁₁-X₁₂-Leu-X₁₃-X₁₄-Leu-Ile-Ala-Glu-
Ile-His-Thr-Ala-Glu-X₁₅-Cys,

where

X₀₁ is Met, Leu, or Nle;

X₀₂ is Asn, Ala, Val, Asp, Glu, or Gln;

X₀₃ is Leu, Ala, Val, Met, Lys, Ile, Arg, Har, or Trp;

X₀₄ is Gly, Ala, His, Arg, or dTrp;

X₀₅ is Lys, Ala, Leu, Gln, Arg, His, or Trp;

X₀₆ is His, Leu, Arg, Phe, Trp, or Ala;

X₀₇ is Ile or Leu;

X₀₈ is Gln or Asn;

X₀₉ is Asp or Ser;

X₁₀ is Ala, Leu, Met, Glu, Ser, or Phe;

X₁₁ is Ala, Phe, Glu, Ser, Leu, Asn, Trp, or Lys;

X₁₂ is Phe or Trp;

X₁₃ is His, Arg, Leu, Trp, or Lys;

X₁₄ is Lys, His, Ala, Ser, Asn, or Arg; and

X₁₅ is Ile, Cys, or Tyr;

or a fragment thereof containing from 24 to 32 contiguous amino acid residues of formula (I).

In certain embodiments, the polypeptide is a fragment of the N-terminally truncated PTH/PTHrP hybrid peptide containing amino acid residues 1-32 of formula (I). In further embodiments, the polypeptide is a fragment of the N-terminally truncated PTH/PTHrP hybrid peptide containing amino acid residues 3-32 of formula (I). In particular embodiments, the polypeptide is a fragment of the N-terminally truncated PTH/PTHrP hybrid peptide containing amino acid residues 3-33 of formula (I).

In further embodiments, X₀₁ is Met, X₀₄ is Ala, X₁₂ is Phe, and X₁₅ is Ile. In yet further embodiments, X₀₁ is Met, X₀₄ is dTrp, X₁₂ is Trp, and X₁₅ is Ile. In still further embodiments, X₀₁ is Nle, X₀₄ is dTrp, X₁₂ is Trp, and X₁₅ is Tyr. In some embodiments, X₀₁ is Nle, X₀₄ is dTrp, X₁₂ is Trp, and X₁₅ is Cys. In certain embodiments, X₀₁ is Nle, X₀₄ is dTrp, X₁₂ is Trp, and X₁₅ is Ile. In particular embodiments, X₀₁ is Met, X₀₄ is dTrp, X₁₂ is Trp, and X₁₅ is Ile. In some embodiments, X₀₄ is dTrp. In certain embodiments, X₁₂ is Trp.

In other embodiments, the N-terminally truncated PTH/PTHrP hybrid peptide has the amino acid sequence

(SEQ ID NO: 1)
Ile-Gln-Leu-Met-His-Gln-Arg-Ala-Lys-Trp-Ile-Gln-
Asp-Ala-Arg-Arg-Arg-Ala-Phe-Leu-His-Lys-Leu-Ile-
Ala-Glu-Ile-His-Thr-Ala-Glu-Ile;
(SEQ ID NO: 2)
Ile-Gln-Leu-Met-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile;
(SEQ ID NO: 3)
Ile-Gln-Leu-Nle-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Tyr;
(SEQ ID NO: 4)
Ile-Gln-Leu-Nle-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Cys;
(SEQ ID NO: 5)
Ile-Gln-Leu-Nle-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile-Cys;
(SEQ ID NO: 6)
Ile-Gln-Leu-Met-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile-Cys;
(SEQ ID NO: 14)
Leu-Met-His-Gln-Arg-dTrp-Lys-Trp-Ile-Gln-Asp-Ala-
Arg-Arg-Arg-Ala-Trp-Leu-His-Lys-Leu-Ile-Ala-Glu-
Ile-His-Thr-Ala-Glu-Ile;
(SEQ ID NO: 15)
Leu-Nle-His-Gln-Arg-dTrp-Lys-Trp-Ile-Gln-Asp-Ala-
Arg-Arg-Arg-Ala-Trp-Leu-His-Lys-Leu-Ile-Ala-Glu-
Ile-His-Thr-Ala-Glu-Ile;
(SEQ ID NO: 16)
Leu-Nle-His-Gln-Leu-dTrp-Lys-Trp-Ile-Gln-Asp-Ala-
Arg-Arg-Arg-Ala-Trp-Leu-His-Lys-Leu-Ile-Ala-Glu-
Ile-His-Thr-Ala-Glu-Ile;
(SEQ ID NO: 17)
Ile-Gln-Leu-Nle-His-Gln-Leu-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile;
(SEQ ID NO: 18)
Leu-Met-His-Gln-Leu-dTrp-Lys-Trp-Ile-Gln-Asp-Ala-
Arg-Arg-Arg-Ala-Trp-Leu-His-Lys-Leu-Ile-Ala-Glu-
Ile-His-Thr-Ala-Glu-Ile;
or
(SEQ ID NO: 19)
Ile-Gln-Leu-Met-His-Gln-Leu-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile;

or a 3-32 or 3-33 fragment thereof.

In yet other embodiments, the polypeptide contains a radionuclide, a polyethylene glycol, or a dye.

In another aspect, the invention provides a pharmaceutical composition containing the polypeptide of the invention and a pharmaceutically acceptable carrier.

In yet another aspect, the invention provides a method of antagonizing or inversely agonizing the activity of parathyroid hormone receptor 1 (PTHR1) in a cell by contacting the cell with the polypeptide of the invention.

In some embodiments, the cell is a human cell.

In a further aspect, the invention provides a method of treating a disease or condition associated with PTHR1 signaling overactivity by administering to the subject an effective amount of the polypeptide of the invention or the pharmaceutical composition of the invention.

In certain embodiments, the disease or condition is hypercalcemia, hypophosphatemia, hyperparathyroidism, or Jansen's chondrodysplasia. In particular embodiments, the administering involves subcutaneous, intravenous, intranasal, transpulmonary, transdermal, transmucosal, or oral administration of the polypeptide or the pharmaceutical composition to the subject.

The invention is also described by the following items.

1. A polypeptide or a pharmaceutically acceptable salt thereof comprising an N-terminally truncated PTH/PTHrP hybrid peptide or a fragment thereof, wherein said polypeptide is a PTHR1 antagonist or a PTHR1 inverse agonist.

2. The polypeptide of item 1, wherein the N-terminally truncated PTH/PTHrP hybrid peptide is of formula (I):

(I)
Ile-Gln-Leu-X₀₁-His-X₀₂-X₀₃-X₀₄-X₀₅-X₀₆-X₀₇-X₀₈-X₀₉-
X₁₀-Arg-Arg-Arg-X₁₁-X₁₂-Leu-X₁₃-X₁₄-Leu-Ile-Ala-Glu-
Ile-His-Thr-Ala-Glu-X₁₅-Cys,

wherein

X₀₁ is Met, Leu, or Nle;

X₀₂ is Asn, Ala, Val, Asp, Glu, or Gln;

X₀₃ is Leu, Ala, Val, Met, Lys, Ile, Arg, Har, or Trp;

X₀₄ is Gly, Ala, His, Arg, or dTrp;

X₀₅ is Lys, Ala, Leu, Gln, Arg, His, or Trp;

X₀₆ is His, Leu, Arg, Phe, Trp, or Ala;

X₀₇ is Ile or Leu;

X₀₈ is Gln or Asn;

X₀₉ is Asp or Ser;

X₁₀ is Ala, Leu, Met, Glu, Ser, or Phe;

X₁₁ is Ala, Phe, Glu, Ser, Leu, Asn, Trp, or Lys;

X₁₂ is Phe or Trp;

X₁₃ is His, Arg, Leu, Trp, or Lys;

X₁₄ is Lys, His, Ala, Ser, Asn, or Arg; and

X₁₅ is Ile, Cys, or Tyr;

or a fragment thereof comprising from 24 to 32 contiguous amino acid residues of formula (I).

3. The polypeptide of item 2, wherein the polypeptide is a fragment of the N-terminally truncated PTH/PTHrP hybrid peptide comprising amino acid residues 1-32 of formula (I).

4. The polypeptide of item 2, wherein the polypeptide is a fragment of the N-terminally truncated PTH/PTHrP hybrid peptide comprising amino acid residues 3-32 of formula (I).

5. The polypeptide of item 2, wherein the polypeptide is a fragment of the N-terminally truncated PTH/PTHrP hybrid peptide comprising amino acid residues 3-33 of formula (I).

6. The polypeptide of any one of items 2 to 5, wherein X₀₁ is Met or Nle.

7. The polypeptide of any one of items 2 to 6, wherein X₀₂ is Asn or Gln.

8. The polypeptide of any one of items 2 to 7, wherein X₀₃ is Leu, Arg, or Har.

9. The polypeptide of any one of items 2 to 8, wherein X₀₅ is Lys.

10. The polypeptide of any one of items 2 to 9, wherein X₀₆ is His or Trp.

11. The polypeptide of any one of items 2 to 10, wherein X₁₀ is Ala, Leu, or Met; X₁₁ is Ala or Phe; X₁₃ is His or Arg; and X₁₄ is Lys or His.

12. The polypeptide of item 11, wherein X₁₀ is Ala, X₁₁ is Ala, X₁₃ is His, and X₁₄ is Lys.

13. The polypeptide of any one of items 2 to 12, wherein X₀₇ is Ile; X₀₈ is Gln; and X₀₉ is Asp.

14. The polypeptide of any one of items 2 to 13, wherein X04 is Gly, Ala, or dTrp.

15. The polypeptide of item 1 or 2, wherein the N-terminally truncated PTH/PTHrP hybrid peptide has the amino acid sequence

(SEQ ID NO: 1)
Ile-Gln-Leu-Met-His-Gln-Arg-Ala-Lys-Trp-Ile-Gln-
Asp-Ala-Arg-Arg-Arg-Ala-Phe-Leu-His-Lys-Leu-Ile-
Ala-Glu-Ile-His-Thr-Ala-Glu-Ile;
(SEQ ID NO: 2)
Ile-Gln-Leu-Met-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile;
(SEQ ID NO: 3)
Ile-Gln-Leu-Nle-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Tyr;
(SEQ ID NO: 4)
Ile-Gln-Leu-Nle-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Cys;
(SEQ ID NO: 5)
Ile-Gln-Leu-Nle-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile-Cys;
(SEQ ID NO: 6)
Ile-Gln-Leu-Met-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile-Cys;
(SEQ ID NO: 14)
Leu-Met-His-Gln-Arg-dTrp-Lys-Trp-Ile-Gln-Asp-Ala-
Arg-Arg-Arg-Ala-Trp-Leu-His-Lys-Leu-Ile-Ala-Glu-
Ile-His-Thr-Ala-Glu-Ile;
(SEQ ID NO: 15)
Leu-Nle-His-Gln-Arg-dTrp-Lys-Trp-Ile-Gln-Asp-Ala-
Arg-Arg-Arg-Ala-Trp-Leu-His-Lys-Leu-Ile-Ala-Glu-
Ile-His-Thr-Ala-Glu-Ile;
(SEQ ID NO: 16)
Leu-Nle-His-Gln-Leu-dTrp-Lys-Trp-Ile-Gln-Asp-Ala-
Arg-Arg-Arg-Ala-Trp-Leu-His-Lys-Leu-Ile-Ala-Glu-
Ile-His-Thr-Ala-Glu-Ile;
(SEQ ID NO: 17)
Ile-Gln-Leu-Nle-His-Gln-Leu-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile;
(SEQ ID NO: 18)
Leu-Met-His-Gln-Leu-dTrp-Lys-Trp-Ile-Gln-Asp-Ala-
Arg-Arg-Arg-Ala-Trp-Leu-His-Lys-Leu-Ile-Ala-Glu-
Ile-His-Thr-Ala-Glu-Ile;
or
(SEQ ID NO: 19)
Ile-Gln-Leu-Met-His-Gln-Leu-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile;

or a 3-32 or 3-33 fragment thereof.

16. The polypeptide of any one of items 1 to 15, further comprising a radionuclide, a polyethylene glycol, or a dye.

17. A pharmaceutical composition comprising the polypeptide of any one of items 1 to 16 and a pharmaceutically acceptable carrier.

18. A method of antagonizing the activity of parathyroid hormone receptor 1 (PTHR1) in a cell, the method comprising contacting the cell with the polypeptide of any one of items 1 to 16, wherein, after the contacting, the activity of PTHR1 in the cell is antagonized.

19. A method of inversely agonizing the activity of parathyroid hormone receptor 1 (PTHR1) in a cell, the method comprising contacting the cell with the polypeptide of any one of items 1 to 16, wherein, after the contacting, the activity of PTHR1 in the cell is inversely agonized.

20. The method of item 18 or 19, wherein the cell is a human cell.

21. A method of treating a disease or condition associated with a PTHR1 signaling overactivity, the method comprising administering to the subject an effective amount of the polypeptide of any one of items 1 to 16 or the pharmaceutical composition of item 17.

22. A method of treating hypercalcemia in a subject, the method comprising administering to the subject an effective amount of the polypeptide of any one of items 1 to 16 or the pharmaceutical composition of item 17.

23. A method of treating hypophosphatemia in a subject, the method comprising administering to the subject an effective amount of the polypeptide of any one of items 1 to 16 or the pharmaceutical composition of item 17.

24. A method of treating hyperparathyroidism in a subject, the method comprising administering to the subject an effective amount of the polypeptide of any one of items 1 to 16 or the pharmaceutical composition of item 17.

25. A method of treating Jansen's chondrodysplasia in a subject, the method comprising administering to the subject an effective amount of the polypeptide of any one of items 1 to 16 or the pharmaceutical composition of item 17.

26. The method of item 25, wherein the polypeptide is as defined in any one of items 2 to 16, and wherein Xo4 is dTrp.

27. The method of any one of items 21 to 26, wherein the administering comprises subcutaneous, intravenous, intranasal, transpulmonary, transdermal, transmucosal, or oral administration of the polypeptide or the pharmaceutical composition to the subject.

28. The method of any one of items 21 to 27, wherein the subject is a human.

29. The polypeptide of any one of items 1 to 16 or the pharmaceutical composition of item 17 for treating a disease or condition associated with PTHR1 signaling overactivity in a subject.

30. The polypeptide of any one of items 1 to 16 or the pharmaceutical composition of item 17 for treating hypophosphatemia in a subject.

31. The polypeptide of any one of items 1 to 16 or the pharmaceutical composition of item 17 for treating hyperparathyroidism in a subject.

32. The polypeptide of any one of items 1 to 16 or the pharmaceutical composition of item 17 for treating Jansen's chondrodysplasia in a subject.

33. The polypeptide or the pharmaceutical composition of item 31, wherein the polypeptide is as defined in any one of items 2 to 16, and wherein X₀₄ is dTrp.

34. The pharmaceutical composition of any one of items 29 to 33 formulated for subcutaneous, intravenous, intranasal, transpulmonary, transdermal, transmucosal, or oral administration to the subject.

35. Use of the polypeptide of any one of items 1 to 16 or the pharmaceutical composition of item 17 in the manufacture of a medicament for treating a disease or condition associated with a PTHR1 signaling overactivity in a subject.

36. Use of the polypeptide of any one of items 1 to 16 or the pharmaceutical composition of item 17 in the manufacture of a medicament for treating hypercalcemia in a subject.

37. Use of the polypeptide of any one of items 1 to 16 or the pharmaceutical composition of item 17 in the manufacture of a medicament for treating hypophosphatemia.

38. Use of the polypeptide of any one of items 1 to 16 or the pharmaceutical composition of item 17 in the manufacture of a medicament for treating hyperparathyroidism in a subject.

39. Use of the polypeptide of any one of items 1 to 16 or the pharmaceutical composition of item 17 in the manufacture of a medicament for treating Jansen's chondrodysplasia.

40. The use of item 39, wherein the polypeptide is as defined in any one of times 2 to 16, and wherein X₀₄ is dTrp.

41. The use any one of items 35 to 40, wherein the medicament is formulated for subcutaneous, intravenous, intranasal, transpulmonary, transdermal, transmucosal, or oral administration to the subject.

### Definitions

The term “dye” is used herein to mean an agent known in the art to be useful in the imaging of biological systems (e.g., a fluorescent dye (e.g., tetramethylrhodamine)).

The term “effective amount,” when used in reference to treating a condition or disease (e.g., hypercalcemia, hypophosphatemia, hyperparathyroidism, or Jansen's chondrodysplasia), refers to an amount of a polypeptide of the invention or a pharmaceutically acceptable salt thereof that treats the condition or disease in a subject.

The term “endogenous agonist” of a parathyroid hormone receptor 1 (PTHR1) is used herein to mean a compound produced by an organism, or a synthetic phenocopy of that compound, i.e., a compound having the same pharmacological activity as the endogenous agonist. For example, the native PTH peptide is (1-84), and PTHrP is ˜(1-140) amino acids; phenocopies of these ligands include PTH(1-34) and PTHrP(1-36), respectively. An endogenous agonist is involved in or modulates the normal physiological activation of the PTHR1. PTHR1 has multiple endogenous agonists (e.g., PTH and PTHrP).

The term “fragment,” when used in reference to an N-terminally truncated PTH/PTHrP hybrid peptide, refers to a portion of the N-terminally truncated PTH/PTHrP hybrid peptide. Thus, a 1-n fragment of formula (I) refers to a polypeptide having a sequence that starts at the first N-terminal amino acid residue in formula (I) and ends at the n^{th} amino acid residue in formula (I). Similarly, a 3-n fragment of formula (I) refers to a polypeptide having a sequence that starts at the third N-terminal amino acid residue in formula (I) and ends at the n^{th} amino acid residue in formula (I).

The term “N-terminally truncated PTH/PTHrP hybrid peptide” is used herein to mean a compound including PTH(X-Y)/PTHrP(Z-37) peptide (e.g., hPTH(X-Y)/hPTHrP(Z-37) peptide), where X is from 2 to 7 (e.g., X is from 5 to 7), Y is from 11 to 18 (e.g., Y is 14), and Z is Y+1, where PTH has a sequence of a 34-amino acid residue-long portion of the parathyroid hormone peptide (e.g., hPTH having a sequence of SEQ ID NO:10 and with the numbering starting at the first N-terminal residue of SEQ ID NO:10 for hPTH), and PTHrP having a sequence of a 37-amino acid residue-long portion of the parathyroid hormone related peptide (e.g., hPTHrP having a sequence of SEQ ID NO:11, and with the numbering starting at the first N-terminal residue of SEQ ID NO:11 for PTHrP). The PTH(5-Y)/PTHrP(Z-37) peptide may be wt-hPTH(5-Y)/wt-hPTHrP(Z-37), which, in some embodiments, includes from 1 to 14 amino acid substitutions in the amino acid sequence of wt-hPTH(X-Y)/wt-hPTHrP(Z-37) peptide. Likewise, PTH(7-Y)/PTHrP(Z-37) peptide may be wt-hPTH(7-Y)/wt-HPTHrP(Z-37), which, in some embodiments, includes from 1 to 14 amino acid substitutions in the amino acid sequence of the wt-hPTH(X-Y)/wt-hPTHrP(Z-37) peptide. PTH(X-Y)/PTHrP(Z-37) may be abbreviated herein as LA-PTH(X-37). Similarly, a fragment of PTH(X-Y)/PTHrP(Z-37), in which the 37th amino acid residue is absent, may be abbreviated herein as LA-PTH(X-36).

The terms “polypeptide” and “peptide” are used interchangeably herein to mean a compound that contains a sequence of amino acids bonded to each other through peptidic bonds. A polypeptide or peptide includes at least 10 amino acids.

The term “PTHR1” is used herein to mean a parathyroid hormone receptor 1 (e.g., a human parathyroid hormone receptor 1 (hPTHR1)). PTHR1 may be wild-type or may be a naturally-occurring mutant PTHR1 which has constitutive activity (e.g., PTHR1 expressed in cells of a subject having Jansen's chondrodysplasia). For example, a naturally-occurring mutant PTHR1 which has constitutive activity can be PTHR1-H223R or PTHR1-T410P.

The term “PTHR1 antagonist” is used herein to mean a polypeptide capable of binding PTHR1, thereby blocking or dampening endogenous agonist-mediated responses without agonizing the signaling activity of PTHR1. The activity of PTHR1 antagonist may be assessed using methods known in the art for assessing antagonist activity or using methods described herein.

The term “PTHR1 inverse agonist” is used herein to mean a polypeptide capable of binding PTHR1 having a constitutive activity and, upon binding, reducing the constitutive activity of PTHR1. The activity of PTHR1 inverse agonist may be assessed using methods known in the art for assessing inverse agonist activity or using methods described herein.

The term “radionuclide” is used herein to mean a radioactive isotope known in the art to be useful in imaging of biological systems.

The term “subject” is used herein to mean a mammal (e.g., a human) diagnosed by a medical practitioner as having a condition or disease, e.g., a disease associated with the PTHR1 signaling overactivity (e.g., hypercalcemia, hypophosphatemia, hyperparathyroidism, or Jansen's chondrodysplasia). Diagnosis may be performed by techniques and methods known in the art. A subject to be treated according to the methods of the invention may have been subjected to standard tests (e.g., tests for serum calcium levels or serum phosphate levels) or may have been identified, without such tests, as one at high risk due to the presence of one or more risk factors (e.g., diseases associated with elevated serum calcium levels (e.g., cancer, tuberculosis, and sarcoidosis) and therapeutic regimens increasing the release of parathyroid hormone (e.g., lithium) or reducing serum phosphate levels (e.g., antacids)).

The terms “treating” or “treatment,” when used herein in reference to a subject, are used herein to mean ameliorating at least one symptom of a condition or disease in a subject having the condition or disease (e.g., a subject diagnosed with hyperparathyroidism, hypercalcemia, hypophosphatemia, or Jansen's chondrodysplasia), as compared with an equivalent untreated control. Such reduction in the symptom (e.g., a reduction in serum calcium levels or an increase in serum phosphate levels) is at least 5% (e.g., at least 10%, 20%, 40%, 50%, 60%, 80%, 90%, 95%, or 100%), as measured in accordance with methods recognized in the art as suitable for assessing the symptom (e.g., serum calcium or phosphate levels).

Unless defined otherwise, all technical and scientific terms used herein have the same meaning as commonly understood to one of ordinary skill in the art to which this disclosure belongs. For any term present in the art which is identical to any term expressly defined in this disclosure, the term's definition presented in this disclosure will control in all respects. Although methods and materials similar or equivalent to those described herein can be used in the practice of the disclosed methods and compositions, the exemplary methods and materials are described herein. Other features and advantages of the invention will be apparent from the following Detailed Description, the drawings, and the claims.

### BRIEF DESCRIPTION OF THE DRAWINGS

FIGS. 1A and 1B are graphs showing results for competition binding experiments using ¹²⁵I-PTH(1-34) and exemplary polypeptides.

FIG. 2 is a graph showing results for the assay assessing antagonism of PTH(1-34)-induced cAMP response in GP-2.3 cells.

FIG. 3A is a graph showing results for the assay assessing antagonism of PTH(1-34)-induced cAMP response in SGS-72 cells.

FIG. 3B is a graph showing results for the assay assessing antagonism of PTHrP(1-36)-induced cAMP response in SGS-72 cells.

FIGS. 4A, 4B, 4C, and 4D are graphs showing pre-incubation/pre-washout cAMP levels, as measured by luminescence (GloSensor™), in GP-2.3 cells in response to contacting with exemplary polypeptides.

FIGS. 4E, 4F, 4G, and 4H are graphs showing cAMP levels, as measured by luminescence (GloSensor™), in response to the addition of PTH(1-34) to GP-2.3 cells after the antagonist wash-out from the cell culture pre-treated with exemplary polypeptides.

FIGS. 5A, 5B, 5C, and 5D are graphs showing cAMP levels, as measured by luminescence (GloSensor™), in response to the addition of M-PTH(1-11)-823 to GP-2.3 cells after the antagonist wash-out from the cell culture pre-treated with exemplary polypeptides.

FIGS. 5E, 5F, 5G, and 5H are graphs showing cAMP levels, as measured by luminescence (GloSensor™), in response to the addition of isoproterenol-1839 to GP-2.3 cells after the antagonist wash-out from the cell culture pre-treated with exemplary polypeptides.

FIGS. 6A, 6B, 6C, 6D, and 6E are graphs showing pre-incubation/pre-washout cAMP levels, as measured by luminescence (GloSensor™), in SGS-72 cells in response to contacting with exemplary polypeptides.

FIGS. 6F, 6G, 6H, 6I, and 6J are graphs showing cAMP levels, as measured by luminescence (GloSensor™), in response to the addition of PTH(1-34) to SGS-72 cells after the antagonist wash-out from the cell culture pre-treated with exemplary polypeptides. The units along the X axis are minutes, and the units along the Y axis are (cps) for luminescence.

FIGS. 6K, 6L, 6M, 6N, and 6O are graphs showing cAMP levels, as measured by luminescence (GloSensor™), in response to the addition of M-PTH(1-11)-823 to SGS-72 cells after the antagonist wash-out from the cell culture pre-treated with exemplary polypeptides. The units along the X axis are minutes, and the units along the Y axis are (cps) for luminescence.

FIG. 7A is a graph showing cAMP levels, as measured by luminescence (GloSensor™), in GP-2.3 cells in response to the addition of an exemplary polypeptide or vehicle.

FIG. 7B is a graph showing cAMP levels, as measured by luminescence (GloSensor™), in HEK293 cells (GHR-10 cell line) in response to the addition of an exemplary polypeptide or vehicle.

FIG. 7C is a graph showing cAMP levels, as measured by luminescence (GloSensor™), in HEK293 cells (GTP-4 cell line) in response to the addition of an exemplary polypeptide or vehicle.

FIGS. 8A, 8B, and 8C are pairs of graphs showing PTHR1 binding kinetics for exemplary polypeptides (both on and off graphs are provided) in GP-2.3 cell membranes. The units along the X-axis are minutes.

FIGS. 9A and 9B are graphs showing cAMP levels, as measured by luminescence (GloSensor™) in GP-2.3 cells. These graphs provide results for assays assessing residual agonist activity in the polypeptides disclosed herein.

FIGS. 10A and 10B are images showing that TMR-LA-PTH(5-36)-1953 is retained bound to PTHR1 on the cell surface (FIG. 10B), whereas TMR-PTH(1-35)-1962 is internalized into endosomal vesicles (FIG. 10B).

FIG. 11 is a graph showing blood Ca²⁺ levels over time in mice after intravenous administration of the exemplary polypeptides to the mice.

FIG. 12A is a graph showing blood Ca²⁺ levels in mice (10-week old female CD1 strain) with hyperparathyroid hypercalcemia that was induced by injecting PEG-PTH(1-35)-1925 agonist (50 nmol/kg, i.v.) at −24 h. At 0 h, the mice were injected with vehicle, dW12-PTH(7-34)-1951 (500 nmol/kg), or dW12,LA-PTH(5-36)-1952 (500 nmol/kg), and blood Ca²⁺ levels were measured. Of the tested peptides, dW12,LA-PTH(5-36)-1952 reduced blood Ca²⁺. Data are means±SE; n=6.

FIG. 12B is a graph showing the data normalized to t=0 h, which reveals significance (P<0.05) for the difference between the vehicle and dW12, LA-PTH(5-36)-1952 values at 2 h.

FIG. 13 is a graph showing the effect of LA-PTH(5-36)-1952 in a mouse model of hypercalcemia of malignancy (excessive blood PTHrP). Mice (10-week old female CD1 strain) were co-injected intravenously with PTHrP(1-36)-1880 agonist (10 nmol/kg) and a vehicle, dW12-PTH(7-34)-1951 (500 nmol/kg), or dW12,LA-PTH(5-36)-1952 (500 nmol/kg). Blood Ca²⁺ levels were measured. Of the tested peptides, administration of dW12,LA-PTH(5-36)-1952 led to a significant reduction of blood Ca²⁺ levels. Data are means±SE; n=6.

FIG. 14A is a graph showing the blood Ca²⁺ levels in mice (9-week old female CD1 strain) injected with vehicle, vehicle+PTH(1-34)-1923 (10 nmol/kg), or PTH(1-34)-1923 (10 nmol/kg)+PEG-LA-PTH(5-37)-1978 (30 nmol/kg). Data are means±SE; n=5 (vehicle), n=6 (PTH+vehicle), n=4 (PTH+PEG-LA-PTH(5-37)-1978).

FIG. 14B is a graph showing TMR-ligand concentration, as measured by fluorescence in plasma from mice (9-week old female CD1 strain) after injected with PEG-LA-PTH(5-37)-1978 (30 nmol/kg) or LA-PTH(5-37)-1975 (30 nmol/kg). Data are means±SE; n=3. t_{1/2}=0.9 h for PEG-LA-PTH(5-37)-1978, t_{1/2} was not determined for LA-PTH(5-37)-1975.

FIG. 15A is a graph showing cAMP response of GP-2.3 cells expressing WT-PTHR to vehicle, dW12-LA-PTH(7-36)-1990, or Nle8,dW12-LA-PTH(7-36)-1992. Units along the vertical axis are counts per second. Units along the horizontal axis are minutes.

FIG. 15B is a graph showing cAMP response of GP-2.3 cells expressing WT-PTHR to agonist PTH(1-34). Units along the vertical axis are counts per second. Units along the horizontal axis are minutes.

FIG. 15C is a graph showing cAMP response of GHR-10 cells expressing PTHR-H223R to vehicle, dW12-LA-PTH(7-36)-1990, or Nle8,dW12-LA-PTH(7-36)-1992. Units along the vertical axis are counts per second. Units along the horizontal axis are minutes.

FIG. 15D is a graph showing cAMP response of GHR-10 cells expressing PTHR-H223R to agonist PTH(1-34). Units along the vertical axis are counts per second. Units along the horizontal axis are minutes.

FIG. 15E is a graph showing cAMP response of GTP-4 cells expressing PTHR-T410P to vehicle, dW12-LA-PTH(7-36)-1990, or Nle8,dW12-LA-PTH(7-36)-1992. Units along the vertical axis are counts per second. Units along the horizontal axis are minutes.

FIG. 15F is a graph showing cAMP response of GTP-4 cells expressing PTHR-T410P to agonist PTH(1-34). Units along the vertical axis are counts per second. Units along the horizontal axis are minutes.

FIG. 16A is a graph showing cAMP response, as measured by GloSensor™ luminescence, of GHR-10 cells to dW12-LA-PTH(5-36)-1952. Units along the vertical axis are counts per second. Units along the horizontal axis are minutes. In FIG. 16A, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 16B is a graph showing cAMP levels, as measured by GloSensor™ luminescence, of GHR-10 cells after washout of dW12-LA-PTH(5-36)-1952. Units along the vertical axis are counts per second. Units along the horizontal axis are minutes. In FIG. 16B, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 16C is a graph showing cAMP response, as measured by GloSensor™ luminescence, of GHR-10 cells to PTH(1-34) after the washout of dW12-LA-PTH(5-36)-1952. Units along the vertical axis are counts per second. Units along the horizontal axis are minutes. In FIG. 16C, pre-9 indicates treatment of the cells with 1×10⁻⁹ M peptide, pre-8 indicates treatment of the cells with 1×10⁻⁸ M peptide, pre-7 indicates treatment of the cells with 1×10⁻⁷ M peptide, and pre-6 indicates treatment of the cells with 1×10⁻⁶ M peptide.

FIG. 17A is a graph showing cAMP response, as measured by GloSensor™ luminescence, of GHR-10 cells to dW12-LA-PTH(7-36)-1990. Units along the vertical axis are counts per second. Units along the horizontal axis are minutes. In FIG. 17A, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 17B is a graph showing cAMP levels, as measured by GloSensor™ luminescence, of GHR-10 cells after washout of dW12-LA-PTH(7-36)-1990. Units along the vertical axis are counts per second. Units along the horizontal axis are minutes. In FIG. 17B, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 17C is a graph showing cAMP response, as measured by GloSensor™ luminescence, of GHR-10 cells to PTH(1-34) after the washout of dW12-LA-PTH(7-36)-1990. Units along the vertical axis are counts per second. Units along the horizontal axis are minutes. In FIG. 17C, pre-9 indicates treatment of the cells with 1×10⁻⁹ M peptide, pre-8 indicates treatment of the cells with 1×10⁻⁸ M peptide, pre-7 indicates treatment of the cells with 1×10⁻⁷ M peptide, and pre-6 indicates treatment of the cells with 1×10⁻⁶ M peptide.

FIG. 18A is a graph showing cAMP response, as measured by GloSensor™ luminescence, of GHR-10 cells to Nle8,dW12-LA-PTH(7-36)-1992. Units along the vertical axis are counts per second. Units along the horizontal axis are minutes. In FIG. 18A, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 18B is a graph showing cAMP levels, as measured by GloSensor™ luminescence, of GHR-10 cells after washout of Nle8,dW12-LA-PTH(7-36)-1992. Units along the vertical axis are counts per second. Units along the horizontal axis are minutes. In FIG. 18B, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 18C is a graph showing cAMP response, as measured by GloSensor™ luminescence, of GHR-10 cells to PTH(1-34) after the washout of Nle8,dW12-LA-PTH(7-36)-1992. Units along the vertical axis are counts per second. Units along the horizontal axis are minutes. In FIG. 18C, pre-9 indicates treatment of the cells with 1×10⁻⁹ M peptide, pre-8 indicates treatment of the cells with 1×10⁻⁸ M peptide, pre-7 indicates treatment of the cells with 1×10⁻⁷ M peptide, and pre-6 indicates treatment of the cells with 1×10⁻⁶ M peptide.

FIG. 19A is a graph showing cAMP response, as measured by GloSensor™ luminescence, of GHR-10 cells to Nle8, L11, dW12-LA-PTH(7-36)-1997. Units along the vertical axis are counts per second. Units along the horizontal axis are minutes. In FIG. 19A, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 19B is a graph showing cAMP levels, as measured by GloSensor™ luminescence, of GHR-10 cells after washout of Nle8,L11,dW12-LA-PTH(7-36)-1997. Units along the vertical axis are counts per second. Units along the horizontal axis are minutes. In FIG. 19B, −9 indicates treatment of the cells with 1×10⁻⁹ M peptide, −8 indicates treatment of the cells with 1×10⁻M peptide, −7 indicates treatment of the cells with 1×10⁻⁷ M peptide, and −6 indicates treatment of the cells with 1×10⁻⁶ M peptide.

FIG. 19C is a graph showing cAMP response, as measured by GloSensor™ luminescence, of GHR-10 cells to PTH(1-34) after the washout of Nle8,L11,dW12-LA-PTH(7-36)-1997. Units along the vertical axis are counts per second. Units along the horizontal axis are minutes. In FIG. 19C, pre-9 indicates treatment of the cells with 1×10⁻⁹ M peptide, pre-8 indicates treatment of the cells with 1×10⁻⁸ M peptide, pre-7 indicates treatment of the cells with 1×10⁻⁷ M peptide, and pre-6 indicates treatment of the cells with 1×10⁻⁶ M peptide.

FIG. 20A is a graph showing cAMP response, as measured by GloSensor™ luminescence, of GHR-10 cells to Nle8, L11, dW12-LA-PTH(5-36)-1999. Units along the vertical axis are counts per second. Units along the horizontal axis are minutes. In FIG. 20A, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 20B is a graph showing cAMP levels, as measured by GloSensor™ luminescence, of GHR-10 cells after washout of Nle8, L11, dW12-LA-PTH(5-36)-1999. Units along the vertical axis are counts per second. Units along the horizontal axis are minutes. In FIG. 20B, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 20C is a graph showing cAMP response, as measured by GloSensor™ luminescence, of GHR-10 cells to PTH(1-34) after the washout of Nle8, L11, dW12-LA-PTH(5-36)-1999. Units along the vertical axis are counts per second. Units along the horizontal axis are minutes. In FIG. 20C, pre-9 indicates treatment of the cells with 1×10⁻⁹ M peptide, pre-8 indicates treatment of the cells with 1×10⁻⁸ M peptide, pre-7 indicates treatment of the cells with 1×10⁻⁷ M peptide, and pre-6 indicates treatment of the cells with 1×10⁻⁶ M peptide.

FIG. 21A is a graph showing cAMP response, as measured by GloSensor™ luminescence, of GHR-10 cells to L11, dW12-LA-PTH(7-36)-2001. Units along the vertical axis are counts per second. Units along the horizontal axis are minutes. In FIG. 21A, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 21B is a graph showing cAMP levels, as measured by GloSensor™ luminescence, of GHR-10 cells after washout of L11, dW12-LA-PTH(7-36)-2001. Units along the vertical axis are counts per second. Units along the horizontal axis are minutes. In FIG. 21B, −9 indicates treatment of the cells with 1×10⁻⁹ M peptide, −6 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 21C is a graph showing cAMP response, as measured by GloSensor™ luminescence, of GHR-10 cells to PTH(1-34) after the washout of L11, dW12-LA-PTH(7-36)-2001. Units along the vertical axis are counts per second. Units along the horizontal axis are minutes. In FIG. 21C, pre-9 indicates treatment of the cells with 1×10⁻⁹ M peptide, pre-8 indicates treatment of the cells with 1×10⁻⁸ M peptide, pre-7 indicates treatment of the cells with 1×10⁻⁷ M peptide, and pre-6 indicates treatment of the cells with 1×10⁻⁶ M peptide.

FIG. 22A is a graph showing cAMP response, as measured by GloSensor™ luminescence, of GHR-10 cells to L11, dW12-LA-PTH(5-36)-2003. Units along the vertical axis are counts per second. Units along the horizontal axis are minutes. In FIG. 22A, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 22B is a graph showing cAMP levels, as measured by GloSensor™ luminescence, of GHR-10 cells after washout of L11, dW12-LA-PTH(5-36)-2003. Units along the vertical axis are counts per second. Units along the horizontal axis are minutes. In FIG. 22B, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 22C is a graph showing cAMP response, as measured by GloSensor™ luminescence, of GHR-10 cells to PTH(1-34) after the washout of L11, dW12-LA-PTH(5-36)-2003. Units along the vertical axis are counts per second. Units along the horizontal axis are minutes. In FIG. 22C, pre-9 indicates treatment of the cells with 1×10⁻⁹ M peptide, pre-8 indicates treatment of the cells with 1×10⁻⁸ M peptide, pre-7 indicates treatment of the cells with 1×10⁻⁷ M peptide, and pre-6 indicates treatment of the cells with 1×10⁻⁶ M peptide.

FIG. 23A is a graph showing cAMP response, as measured by GloSensor™ luminescence, of GHR-10 cells to LA-PTH(5-36)-2012. Units along the vertical axis are counts per second. Units along the horizontal axis are minutes. In FIG. 23A, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 23B is a graph showing cAMP levels, as measured by GloSensor™ luminescence, of GHR-10 cells after washout of LA-PTH(5-36)-2012. Units along the vertical axis are counts per second. Units along the horizontal axis are minutes. In FIG. 23B, −9 indicates treatment of the cells with 1×10⁻⁹ M peptide, −8 indicates treatment of the cells with 1×10⁻⁸ M peptide, −7 indicates treatment of the cells with 1×10⁻⁷ M peptide, and −6 indicates treatment of the cells with 1×10⁻⁶ M peptide.

FIG. 23C is a graph showing cAMP response, as measured by GloSensor™ luminescence, of GHR-10 cells to PTH(1-34) after the washout of LA-PTH(5-36)-2012. Units along the vertical axis are counts per second. Units along the horizontal axis are minutes. In FIG. 23C, pre-9 indicates treatment of the cells with 1×10⁻⁹ M peptide, pre-8 indicates treatment of the cells with 1×10⁻⁸ M peptide, pre-7 indicates treatment of the cells with 1×10⁻⁷ M peptide, and pre-6 indicates treatment of the cells with 1×10⁻⁶ M peptide.

FIG. 24A is a drawing showing the timeline for the in vivo testing of peptides in Col1-H223R mice.

FIG. 24B is an image of H&E stained tibiae from wild-type mice that were administered vehicle.

FIG. 24C is an image of H&E stained tibiae from Col1-H223R mice that were administered vehicle.

FIG. 24D is an image of H&E stained tibiae from wild-type mice that were administered dW12-PTHrP(7-36)-2018.

FIG. 24E is an image of H&E stained tibiae from wild-type mice that were administered dW12-PTH(5-36)-1952.

FIG. 24F is a micro CT image of femurs from Col1-H223R mice that were administered vehicle, dW12-PTHrP(7-36)-2018, or dW12-LA-PTH(5-36)-1952.

FIG. 25A is a bar chart showing total serum Ca²⁺ levels in wild-type mice that were administered vehicle (n=8), dW12-PTHrP(7-36)-2018 (n=9), or dW12-LA-PTH(5-36)-1952 (n=5) and in Col1-H223R mice that were administered vehicle (n=10), dW12-PTHrP(7-36)-2018 (n=10), or dW12-LA-PTH(5-36)-1952 (n=1).

FIG. 25B is a bar chart showing total serum phosphate (Pi) levels in wild-type mice that were administered vehicle (n=8), dW12-PTHrP(7-36)-2018 (n=9), or dW12-LA-PTH(5-36)-1952 (n=5) and in Col1-H223R mice that were administered vehicle (n=10), dW12-PTHrP(7-36)-2018 (n=10), or dW12-LA-PTH(5-36)-1952 (n=1).

FIG. 25C is a bar chart showing urinary Ca/Cre levels in wild-type mice that were administered vehicle (n=8), dW12-PTHrP(7-36)-2018 (n=9), or dW12-LA-PTH(5-36)-1952 (n=5) and in Col1-H223R mice that were administered vehicle (n=10), dW12-PTHrP(7-36)-2018 (n=10), or dW12-LA-PTH(5-36)-1952 (n=1).

FIG. 25D is a bar chart showing urinary Pi/Cre levels in wild-type mice that were administered vehicle (n=8), dW12-PTHrP(7-36)-2018 (n=9), or dW12-LA-PTH(5-36)-1952 (n=5) and in Col1-H223R mice that were administered vehicle (n=10), dW12-PTHrP(7-36)-2018 (n=10), or dW12-LA-PTH(5-36)-1952 (n=1).

FIG. 25E is a bar chart showing collagen-1a1 mRNA levels in femurs from wild-type mice that were administered vehicle (n=8), dW12-PTHrP(7-36)-2018 (n=9), or dW12-LA-PTH(5-36)-1952 (n=5) and from Col1-H223R mice that were administered vehicle (n=10), dW12-PTHrP(7-36)-2018 (n=10), or dW12-LA-PTH(5-36)-1952 (n=1).

FIG. 25F is a bar chart showing serum CTX levels in wild-type mice that were administered vehicle (n=8), dW12-PTHrP(7-36)-2018 (n=9), or dW12-LA-PTH(5-36)-1952 (n=5) and in Col1-H223R mice that were administered vehicle (n=10), dW12-PTHrP(7-36)-2018 (n=10), or dW12-LA-PTH(5-36)-1952 (n=1).

### DETAILED DESCRIPTION

In general, the present invention provides PTHR1 antagonist or inverse agonist peptides. The PTHR1 antagonist or inverse agonist peptides of the invention can be used in a method of treating a condition or a disease of the PTHR1 signaling overactivity (e.g., hypercalcemia, hypophosphatemia, hyperparathyroidism, and Jansen's chondrodysplasia). These diseases are typically associated with higher than normal serum levels of calcium, with lower than normal serum levels of phosphate, with higher than normal levels of endogenous PTHR1 agonist(s), or with constitutive activity of PTHR1 mutants.

Advantageously, the polypeptides of the invention can have higher affinity for PTHR1 in comparison to previously reported PTH(7-34) peptides. This advantageous property may be present despite the absence of N-terminal residues that are believed to contribute to overall binding affinity of PTH(1-34) and PTHrP(1-36). Without wishing to be bound by theory, this advantageous property of the polypeptides of the invention is due to their reduced rate of dissociation from PTHR1. A further advantageous attribute of the polypeptides of the invention can be in their effecting a prolonged reduction in the activity of PTHR1 in comparison to, e.g., an N-terminally truncated PTH (e.g., (7-34)PTH).

The polypeptides of the invention can contain an N-terminally truncated PTH/PTHrP hybrid peptide of formula (I):

(I)
Ile-Gln-Leu-X₀₁-His-X₀₂-X₀₃-X₀₄-X₀₅-X₀₆-X₀₇-X₀₈-X₀₉-
X₁₀-Arg-Arg-Arg-X₁₁-X₁₂-Leu-X₁₃-X₁₄-Leu-Ile-Ala-Glu-
Ile-His-Thr-Ala-Glu-X₁₅-Cys,

wherein

X₀₁ is Met, Leu, or Nle;

X₀₂ is Asn, Ala, Val, Asp, Glu, or Gln;

X₀₃ is Leu, Ala, Val, Met, Lys, Ile, Arg, Har, or Trp;

X₀₄ is Gly, Ala, His, Arg, or dTrp;

X₀₅ is Lys, Ala, Leu, Gln, Arg, His, or Trp;

X₀₆ is His, Leu, Arg, Phe, Trp, or Ala;

X₀₇ is Ile or Leu;

X₀₈ is Gln or Asn;

X₀₉ is Asp or Ser;

X₁₀ is Ala, Leu, Met, Glu, Ser, or Phe;

X₁₁ is Ala, Phe, Glu, Ser, Leu, Asn, Trp, or Lys;

X₁₂ is Phe or Trp;

X₁₃ is His, Arg, Leu, Trp, or Lys;

X₁₄ is Lys, His, Ala, Ser, Asn, or Arg; and

X₁₅ is Ile, Cys, or Tyr;

or a fragment thereof including from 24 to 32 contiguous amino acid residues of formula (I).

In some embodiments, a fragment of a polypeptide of formula (I) is a peptide including from 30 to 32 contiguous amino acid residues. In certain embodiments, a fragment of a polypeptide of formula (I) is a peptide including amino acid residues 1-32 of formula (I), amino acid residues 3-32 of formula (I), or amino acid residues 3-33 of formula (I).

Exemplary PTHR1 antagonist/inverse agonist peptides are listed in Table 1.

TABLE 1

| SEQ | | | |
|---|---|---|---|
| ID | Ref. | | |
| NO. | No. | Chemical Name | Sequence |
| 1 | 1950 | LA-PTH(5-36) | IQLMHQRAKWIQDARRRAFLHKLIAEIHTAELNH₂ |
| 2 | 1949 | dW12, W23-LA-PTH(5-36) | IQLMHQRdWKWIQDARRRAWLHKLIAEIHTAEI.NH₂ |
| 2 | 1952 | dW12, W23-LA-PTH(5-36) | IQLMHQRdWKWIQDARRRAWLHKLIAEIHTAEI.NH₂ |
| 3 | 1954 | Nle8, dW12, W23, Y36-LA- | IQLNleHQRdWKWIQDARRRAWLHKLIAEIHTAEY.NH₂ |
| | | PTH(5-36) | |
| 4 | 1970 | Nle8, dW12, W23, C36-LA- | IQLNleHQRdWKWIQDARRRAWLHKLIAEIHTAEC.NH₂ |
| | | PTH(5-36) | |
| 5 | 1974 | Nle8, dW12, W23, C37-LA- | IQLNleHQRdWKWIQDARRRAWLHKLIAEIHTAEIC.NH₂ |
| | | PTH(5-37) | |
| 6 | 1976 | TMR-dW12, W23, C37-LA- | IQLMHQRdWK′WIQDARRRAWLHKLIAEIHTAEIC.NH₂ |
| | | PTH(5-37) | |
| 2 | 1953 | TMR-dW12, W23-LA- | IQLMHQRdWK′WIQDARRRAWLHKLIAEIHTAELNH₂ |
| | | PTH(5-36) | |
| 5 | 1975 | Nle8, TMR, dW12, W23, | IQLNleHQRdWK′WIQDARRRAWLHKLIAEIHTAEIC.NH₂ |
| | | C37-LA-PTH(5-37) | |
| 5 | 1978 | PEG20-Nle8, TMR, dW12, | IQLNleHQRdWK′WIQDARRRAWLHKLIAEIHTAEIC′.NH₂ |
| | | W23, C37-LA-PTH(5-37) | |
| 14 | 1990 | dW12, W23-LA-PTH(7-36)- | LMHQRdWKWIQDARRRAWLHKLIAEIHTAEI.NH₂ |
| | | 1990 | |
| 14 | 1991 | TMR-dW12, W23-LA- | LMHQRdWK′WIQDARRRAWLHKLIAEIHTAEI.NH₂ |
| | | PTH(7-36)-1991 | |
| 15 | 1992 | Nle8, dW12, W23-LA- | LNleHQRdWKWIQDARRRAWLHKLIAEIHTAEI.NH₂ |
| | | PTH(7-36)-1992 | |
| 15 | 1993 | TMR-Nle8, dW12, W23-LA- | LNleHQRdWK′WIQDARRRAWLHKLIAEIHTAELNH₂ |
| | | PTH(7-36)-1993 | |
| 16 | 1997 | Nle8,L11, dW12, W23-LA- | LNleHQLdWKWIQDARRRAWLHKLIAEIHTAEI.NH₂ |
| | | PTH(7-36)-1997 | |
| 16 | 1998 | TMR-Nle8, L11, dW12, | LNleHQLdWK′WIQDARRRAWLHKLIAEIHTAELNH₂ |
| | | W23-LA-PTH(7-36)-1998 | |
| 17 | 1999 | Nle8, L11, dW12, W23-LA- | IQLNleHQLdWKWIQDARRRAWLHKLIAEIHTAEI.NH₂ |
| | | PTH(5-36)-1999 | |
| 17 | 2000 | TMR-Nle8, L11, dW12, | IQLNleHQLdWK′WIQDARRRAWLHKLIAEIHTAEI.NH₂ |
| | | W23-LA-PTH(5-36)-2000 | |
| 18 | 2001 | L11, dW12, W23-LA-PTH | LMHQLdWKWIQDARRRAWLHKLIAEIHTAEI.NH₂ |
| | | (7-36)-2001 | |
| 18 | 2002 | TMR-L11, dW12, W23-LA- | LMHQLdWK′WIQDARRRAWLHKLIAEIHTAEI.NH₂ |
| | | PTH(7-36)-2002 | |
| 19 | 2003 | L11, dW12, W23-LA-PTH(5- | IQLMHQLdWKWIQDARRRAWLHKLIAEIHTAEI.NH₂ |
| | | 36)-2003 | |
| 19 | 2004 | TMR-L11, dW12, W23-LA- | IQLMHQLdWK′WIQDARRRAWLHKLIAEIHTAEI.NH₂ |
| | | PTH(5-36)-2004 | |
| 1 | 2013 | TMR-LA-PTH(5-36)-2013 | IQLMHQRAK′WIQDARRRAFLHKLIAEIHTAEI.NH₂ |

In Table 1, dW is dTrp, K′ is Lys conjugated to tetramethyl rhodamine, and C′ stands for Cys conjugated to PEG20. In Table 1, polypeptide LA-PTH(5-36)-1950 is a PTHR1 antagonist, and the remaining polypeptides are antagonists/inverse agonists. In Table 1, polypeptides 1949 and 1952 are duplicates.

### Preparation of Peptides

The polypeptides of the invention are amenable to production by solution- or solid-phase peptide synthesis and by in-situ synthesis using combination chemistry. The solid phase peptide synthesis technique, in particular, has been successfully applied in the production of human PTH and can be used for the production of these compounds (for guidance, see, e.g., Fairwell et al., Biochem. 22:2691, 1983). Success with producing human PTH on a relatively large scale has been reported in Goud et al., J Bone Min Res 6:781, 1991. The peptide chemical synthesis approach generally entails the use of automated synthesizers and appropriate resin as solid phase, to which the C-terminal amino acid of a desired polypeptide is attached. Extension of the peptide in the N-terminal direction is then achieved by successively coupling a suitably protected form of the next desired amino acid, typically using chemical protocols based on amino-protecting groups (e.g., Fmoc-or Boc-based), until synthesis is complete. Protecting groups are then cleaved from the peptide, usually with concomitant cleavage of the peptide from the resin, and the peptide is then isolated and purified using conventional techniques, such as by reversed phase HPLC using appropriate mobile phase (e.g., acetonitrile as solvent and tri-fluoroacetic acid as ion-pairing agent). Such procedures are generally described in numerous publications and reference may be made, for example, to Stewart and Young, “Solid Phase Peptide Synthesis,” 2^{nd} Edition, Pierce Chemical Company, Rockford, Ill. (1984).

Polypeptides of the invention can also be made recombinantly by any method known in the art. Prokaryotic (e.g., bacterial) and eukaryotic (e.g., yeast and mammalian) expression systems can also be used to produce polypeptides of the invention, particularly, where the polypeptide includes only proteinogenic amino acids.

### Polypeptide Modifications

The polypeptides of the invention can include the dTrp12 modification. The dTrp12-modified polypeptides of the invention, in addition to their antagonist activity, can typically function as PTHR1 inverse agonists.

Further modifications may be included in the polypeptides of the invention (e.g., N-terminal or C-terminal modifications). The polypeptides of the invention typically include amino acids with side chains amenable to modification, for example, through ester or thioester formation (e.g., Ser, Thr, Tyr, Glu, and Asp), amide formation (e.g., Lys, Glu, and Asp), ether formation (e.g., Ser, Thr, Cys), or amine formation (e.g., Lys). For example, the polypeptides of the invention can be modified to include, e.g., a dye (e.g., tetramethylrhodamine (TMR)) or polyethylene glycol (PEG). The inclusion of a dye can permit tracking the polypeptide of the invention in cells or in vivo through the use of fluorescence. The inclusion of a polyethylene glycol (PEG) group can enhance pharmacokinetic properties of the polypeptide of the invention.

Any of the polypeptides of the invention may further include a heterologous sequence (a fusion partner), thus forming a fusion protein. The fusion protein may include a fusion partner such as a purification or detection tag, for example, proteins that may be detected directly or indirectly such as green fluorescent protein, hemagglutinin, or alkaline phosphatase), DNA binding domains (for example, GAL4 or LexA), gene activation domains (for example, GAL4 or VP16), purification tags, or secretion signal peptides (e.g., preprotrypsin signal sequence). In other embodiments the fusion partner may be a tag, such as c-myc, poly histidine, or FLAG. Each fusion partner may contain one or more domains, e.g., a preprotrypsin signal sequence and FLAG tag. In other cases, the fusion partner is an Fc protein (e.g., mouse Fc or human Fc).

### Pharmaceutical Compositions

The polypeptides disclosed herein may be formulated in a pharmaceutical composition providing an effective amount of the PTHR1 antagonist or inverse agonist to a subject upon administration. The pharmaceutical compositions of the polypeptides disclosed herein can contain an appropriate amount of a suitable carrier or excipient. The pharmaceutical compositions may contain from 0.1% to 95% (w/v) or (w/w) of the PTHR1 antagonist or inverse agonist. The compositions may be provided in a dosage form that is suitable for parenteral (e.g., subcutaneous, intravenous, intramuscular, intraperitoneal), intranasal, transpulmonary, transdermal, transmucosal, or oral administration. Thus, the composition may be in the form of, e.g., tablets, ampules, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 21St edition, 2005, Ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions may be formulated to release the active compound immediately upon administration or at a predetermined time or time period after administration. The latter types of compositions are generally known as controlled release formulations, which include (i) formulations that create substantially constant concentrations of the polypeptides disclosed herein within the body over an extended period of time; (ii) formulations that after a predetermined lag time create substantially constant concentrations of the polypeptides disclosed herein within the body over an extended period of time; (iii) formulations that sustain the action of the polypeptides disclosed herein during a predetermined time period by maintaining a relatively constant, effective level of the polypeptides disclosed herein in the body with concomitant minimization of undesirable side effects associated with fluctuations in the plasma level of the polypeptides disclosed herein (sawtooth kinetic pattern); (iv) formulations that localize action of the polypeptides disclosed herein, e.g., spatial placement of a controlled release composition adjacent to or in the diseased tissue or organ; (v) formulations that achieve convenience of dosing, e.g., administering the composition once per week or once every two weeks; and (vi) formulations that target the action of the polypeptides disclosed herein by using carriers or chemical derivatives to deliver the compound to a particular target cell type. Administration of the compound in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound is formulated with appropriate excipients into a pharmaceutical composition that, upon administration, releases the compound in a controlled manner. Examples include single or multiple unit tablet or capsule compositions, oil solutions, suspensions, emulsions, microcapsules, molecular complexes, microspheres, nanoparticles, patches, and liposomes.

The composition containing polypeptides described herein may be administered parenterally by injection, infusion, or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Compositions for parenteral use may be provided in unit dosage forms (e.g., in single-dose ampoules), in vials containing several doses and in which a suitable preservative may be added, or in prefilled syringes. The composition may be in the form of a solution, a suspension, an emulsion, an infusion device, or a delivery device for implantation, or it may be presented as a dry powder to be reconstituted with water or another suitable vehicle before use. In addition to the polypeptides disclosed herein, the composition may include suitable parenterally acceptable carriers and/or excipients. The polypeptides disclosed herein may be incorporated into microspheres, microcapsules, nanoparticles, or liposomes for controlled release. Furthermore, the composition may include suspending, solubilizing, stabilizing, pH-adjusting agents, tonicity adjusting agents, and/or dispersing agents.

As indicated above, the pharmaceutical compositions according to the invention may be in a form suitable for sterile injection. To prepare such a composition, the suitable the polypeptides disclosed herein are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation may also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Methods of Treatment

The polypeptides and the pharmaceutical compositions disclosed herein may be used to treat a condition or a disease of the PTHR1 signaling overactivity (e.g., hypercalcemia, hypophosphatemia, hyperparathyroidism, and Jansen's chondrodysplasia). PTHR1 signaling overactivity may be caused by various factors, such as elevated blood levels of PTH (e.g., hyperparathyroidism) or PTHrP (e.g., humoral hypercalcemia of malignancy).

Some forms of hypercalcemia are related to the interaction of PTHR1 with PTH or PTHrP (e.g., humoral hypercalcemia of malignancy). Hypercalcemia is a condition in which there is an abnormal elevation in serum calcium levels; it is often associated with other diseases, including hyperparathyroidism, osteoporosis, and cancer (e.g., carcinomas of the breast, lung and prostate, epidermoid cancers of the head and neck and of the esophagus, multiple myeloma, and hypernephroma).

Jansen's chondrodysplasia is a rare disease caused by PTHR1 activating mutations (e.g. H223R and T410P) which result in excessive hormone-independent (constitutive) signaling by the receptor itself. Ligands that bind to such constitutively active PTHR1 mutants and suppress its signaling are classified as PTHR1 inverse agonists. Some, but not all, ligands that function as PTHR1 antagonists also function as PTHR1 inverse agonists. Typically, among the polypeptides disclosed herein, those including a dTrp12 modification function as inverse agonists.

In accordance with yet a further aspect of the invention, there is provided a method for treating a disease or condition that is caused by overactivity of PTHR1 in a subject. The method involves administering to the subject an effective amount of the polypeptide of the invention or a pharmaceutically acceptable salt thereof or a fragment thereof or a pharmaceutical composition disclosed herein. The effective amount will typically be sufficient to reduce activation of the PTHR1 of the subject to non-pathological levels, as assessed by the treatment of the subject.

In one embodiment, a subject having a disease or condition that is caused by the constitutive signaling activity of PTHR1 (e.g., Jansen' chondrodysplasia) can be treated using polypeptides of the invention which are PTHR1 inverse agonists. In this embodiment, the PTHR1 inverse agonist polypeptide of the invention may be present as a pharmaceutically acceptable salt thereof or a fragment thereof or in a pharmaceutical composition disclosed herein.

In another embodiment, a subject having a disease or condition that is caused by the non-constitutive signaling overactivity of PTHR1 can be treated using polypeptides of the invention which are PTHR1 antagonists. In this embodiment, the PTHR1 antagonist polypeptide of the invention may be present as a pharmaceutically acceptable salt thereof or a fragment thereof or in a pharmaceutical composition disclosed herein.

To administer the polypeptide of the invention, the appropriate polypeptide of the invention or a pharmaceutically acceptable salt thereof or a fragment thereof can be used in the manufacture of a medicament, generally by being formulated in an appropriate carrier or excipient such as, e.g., physiological saline, and administered through an appropriate route of administration (e.g., parenteral (e.g., subcutaneous, intravenous, intramuscular, intraperitoneal), intranasal, transpulmonary, transdermal, transmucosal, or oral administration). An effective amount of the polypeptide of the invention is typically present in the medicament. For example, typical dosage would be 1 ng to 10 mg of the polypeptide, e.g., per kg body weight, e.g., per day.

### Methods of Modulating the Activity

The polypeptides disclosed herein may be used to modulate the activity of PTHR1 in a cell. Thus, the present invention features a method of inversely agonizing the activity of PTHR1 in a cell and a method of antagonizing the activity of PTHR1 in a cell. The method may involve contacting the cell with the polypeptide having a desirable activity (e.g., a PTHR1 antagonist or PTHR1 inverse agonist activity). The polypeptide that is a PTHR1 antagonist may be used in this method to antagonizing the signaling activity of PTHR1 in a cell (e.g., by reducing the binding of endogenous agonists to PTHR1). The polypeptide that is a PTHR1 inverse agonist may be used in this method to inversely agonize the constitutive signaling activity of a naturally occurring PTHR1 mutant having constitutive signaling activity (e.g., PTHR1-H223R or PTHR1-T410P). The cell may be in a mammal (e.g., in a subject).

It will be appreciated to those skilled in the art that the invention can be performed within a wide range of equivalent parameters of composition, concentration, modes of administration, and conditions without departing from the spirit or scope of the invention or any embodiment thereof. The following examples are meant to illustrate the invention. They are not meant to limit the invention in any way.

### EXAMPLES

Peptides and Synthesis:

PTH peptides were based on the human PTH(1-34) sequence and were synthesized by the Massachusetts General Hospital Biopolymer Core facility using solid-phase chemistry. Lys¹³(TMR) derivatives were obtained by post-synthetically attaching a fluorescent tetramethylrhodamine (TMR) group to the epsilon amino function of Lys-13. Pegylated derivatives were obtained by post-synthetically attaching a single PEG group (Mw=20,000) to the side chain thiol of a C-terminal cysteine at position 35.

The 20-kDa thiol-reactive PEG reagent, α-[3-(3-Maleimido-1-oxopropyl)amino]propyl-ω-methoxy, polyoxyethylene, SUNBRIGHT® ME-200MA0B, was obtained from NOF America Corp. (White Plains, N.Y.). The thiol conjugation reaction was performed overnight at room temperature in 100 mM sodium citrate buffer, pH 4.0 containing 1 mM EDTA and 10 mM TCEP reducing agent. Unconjugated PEG reagent was removed from the reaction by cation exchange chromatography using SP-sepharose resin, a linear salt gradient was formed using 20 mM sodium acetate pH 4.0 as buffer A, and the same buffer containing 1 M NaCl was used as buffer B. Elution of the PEG-PTH^{TMR} peptide was monitored by measuring the fractions for TMR absorbance at 543 nm. The peak fractions were pooled and de-salted using a C2tp-reverse-phase cartridge and 75% acetonitrile/0.1% TFA for peptide elution. The eluted sample was then lyophilized, and reconstituted in 10 mM acetic at a final ligand concentration of 12.3 mg/ml (0.5 mM); aliquots of these stock solutions were stored at −80° C. until needed for experiments.

All other non-pegylated PTH peptides were purified by reverse phase HPLC and assessed for quality and purity by analytical HPLC and MALDI mass spectrometry; purity was at least 90%. Peptide concentrations of stock solutions were validated by acid hydrolysis and amino acid analysis.

Other peptides of the invention (e.g., LA-PTH(7-36) or LA-PTH(7-37) (e.g., fragments having 3-32 or 3-33 amino acid residues of SEQ ID NOs: 1-6)) can be prepared using methods known in the art (e.g., the exemplary methods described herein).

### Competition Binding Assays:

Ligand binding to the PTH1 R was assessed in intact GP-2.3 cells or membranes prepared from GP-2.3 cells using a ¹²⁵I-PTH peptide analog as tracer radioligand and were incubated at room temperature for 90 minutes. Intact cell binding reactions were performed in 96-well plates, and, following incubation, the cells were lysed with 1N NaOH, and the lysate was counted for gamma radiation. For membrane dissociation assays, reactions were assembled in 15 cc tubes, and, at times following membrane addition (t=0), aliquots were withdrawn and filtered through a well of a vacuum filtration 96-well plate, rinsed, and, after washing, the filters were removed and counted for gamma irradiation. Nonspecific binding was determined in reactions containing an excess (5×10⁻⁷ M) of unlabeled M-PTH(1-15) or PTH(1-34). Curves were fit to the data using a four-parameter sigmoidal dose-response equation.

cAMP Signaling Properties of the Polypeptides in Cells:

cAMP signaling was assessed in the HEK293-derived or SaOS2-derived cell lines stably expressing the luciferase-based GloSensor™ cAMP reporter (Hattersley et al., Paper presented at: Novel Signaling Mechanisms and Bone Cell Biology 2014; Binkowski et al., Methods in molecular biology. 756:263-271). HEK-293 cells were further transfected to stably express the hPTHR1, hPTHR1-H223R, or hPTHR1-T410P. The cells were seeded into 96-well white plates and were assayed 24 to 48 hours post-confluency. Assays were performed at room temperature in CO₂-independent culture media (Life Technologies, Corp., Carlsbad, Calif.) containing 0.1% BSA (CIDB). The cells were pre-loaded with luciferin (0.5 mM in CIDB) for 15 minutes, then PTH peptides were added at varying concentrations. cAMP-dependent luminescence was measured at two-minute intervals using a PerkinElmer Envision plate reader. The time at which maximum luminescence (cps) observed with agonist alone, typically at 10-20 minutes after agonist addition, was used to obtain data from wells containing antagonist with or without agonist, to thus generate antagonist ligand dose-response curves. The resulting cps values were then plotted against ligand concentration using GraphPad Prism 7.0 software and a four-parameter logistics curve fitting equation, which yielded parameters of inhibitory potency (pIC50).

For “wash-out” experiments, the cells pre-loaded with luciferin were treated with media (vehicle) or a test antagonist ligand for 25 minutes; the plate was then removed from the plate reader, the cells were rinsed thrice to remove unbound ligand, and treated with fresh media containing luciferin with or without an agonist ligand. The development of cAMP-dependent luminescence was measured for another 120 minutes.

### In Vivo Testing of the Polypeptides:

Wilde-type, ca. 10-week old male C57BL/6J mice were purchased from the Charles River Laboratories (Wilmington, Mass., USA). The origin and method of breeding of Col1-H223R “Jansen's” transgenic mice is described by Calvi et al. (J. Clin. Invest., 107:277-286, 2001). Mice were maintained in facilities operated by the Center for Comparative Research of the Massachusetts General Hospital, and acclimated in the facilities for seven days prior to being used for study. All experimental procedures were approved by the MGH Institutional Animal Care and Use Committee (IACUC). In each study, animals were assigned randomly to treatment groups. Where possible, power calculations established that the number of animals used per study group was sufficient to detect statistically significant differences in intended primary experimental outcomes (i.e., changes in serum Ca and Pi).

Mice were injected IV via the tail vein or subcutaneously with ligands in vehicle (0.05% Tween80; 10 mM citrate; 150 mM NaCl; pH 5.0) to give the intended final ligand dose (e.g. 50 nmol/kg body weight). At times immediately before (t=0) and after injection, tail vein blood was collected and analyzed for blood ionized calcium (Ca²⁺) measured with a RAPIDLAB 348 analyzer (SIEMENS Healthcare Diagnostic, United Kingdom).

Confocal Microscopy Analysis of PTH-TMR Fluorescence in GP-2.3 cells:

Confocal fluorescence microscopy images were acquired using Olympus FV-1000 MPE confocal system (Center Valley, Pa. 18034), performed by the Photopathology Core facility of the Wellman Center for Photomedicine at the Massachusetts General Hospital. Images were captured with a 40× (LUMPLFL 0.8NA WD 3.3 mm) water-immersion objective, and with 405 nm and 559 nm excitation lasers, at a resolution of 1024×1024.

Ligand internalization properties of PTH^{TMR} analogs were assessed in GP-2.3 cells (HEK-293 with stable transfection of hPTHR1 and GloSensor™); non-specific binding was assessed in GS-22A cells (HEK-293 with stable transfection of GloSensor™; parental to GP-2.3 cells) and was found to be undetectable. The cells were cultured on glass cover-slips in 24-well plates to ˜75% of confluency, then treated with PTH^{TMR} ligand (100 nM) in Hank's balanced salts buffer with 0.1% BSA (HBB) for 15 minutes at room temperature, then rinsed thrice with HBB, fixed with 4% formalin for 5 minutes, mounted with vector-shield containing DAPI on a glass microscope slide, viewed on the microscope, and digitally imaged.

### Data Analysis

Data were processed using Microsoft Excel® and GraphPad Prism 7.0 software packages and analyzed statistically using Student's t test (two-tailed and unequal variances).

### Polypeptides

Polypeptides used in this Example are listed in Tables 1 and 2.

TABLE 2

| SEQ | | | |
|---|---|---|---|
| ID | | | |
| NO | Ref. # | Chemical Name | Sequence |
| 7 | 1894 | dW12, Y34-bPTH(7-34) | FMHNLdWKHLSSMERVEWLRKKLQDVHNY.NH₂ |
| 7 | 1951 | dW12, Y34-bPTH(7-34) | FMHNLdWKHLSSMERVEWLRKKLQDVHNY.NH₂ |
| 8 | 1869 | Nle8, 18, dW12, Y34- | FNleHNLdWKHLSSNleERVEWLRKKLQDVHNY.NH₂ |
| | | bPTH(7-34) (BIM-44002) | |
| 9 | 1977 | Nle8, TMR, dW12, C35- | FNleHNLdWK′HLSSNleERVEWLRKKLQDVHNFC.NH₂ |
| | | bPTH(7-35) | |
| 10 | 1923 | PTH(1-34) | SVSEIQLMHNLGKHLNSMERVEWLRKKLQDVHNF.NH₂ |
| 11 | 1880 | PTHrP(1-36) | AVSEHOLLHDKGKSIQDLRRRFFLHHLIAEIHTAELNH₂ |
| 12 | 823 | M-PTH(1-11) | Ac5cVAibEIQLMHQHar.NH₂ |
| 13 | 1962 | C35, TMR-PTH(1-35).OH | SVSEIQLMHNLGK′HLNSMERVEWLRKKLQDVHNFC.OH |
| 20 | 2018.a | L11, dW12, W23, Y36- | LLHDLdWKSIQDLRRRFWLHHLIAEIHTAEY.NH₂ |
| | | PTHrP(7-36)-2018 | |
| 1 | 2012 | LA-PTH(5-36)-2012 | IQLMHQRAKWIQDARRRAFLHKLIAEIHTAEI.NH₂ |

In Table 2, dW is dTrp; K′ is Lys conjugated to tetramethyl rhodamine; Ac5c is 1-aminocyclopentane-1-carboxylic acid residue; polypeptides 1894, 1951, 1869, and 1977 are antagonists/inverse agonists; and polypeptides 1923, 1880, 823, and 1962 are agonists; 1894 and 1951 are duplicates.

Other polypeptides of the invention may be used in the assays described herein. For example the fragments of the N-terminally truncated PTH/PTHrP hybrid peptide (e.g., LA-PTH(7-36) or LA-PTH(7-37) (e.g., fragments having 3-32 or 3-33 amino acid residues of SEQ ID NOs: 1-6)) may be tested in the assays described herein.

### Results

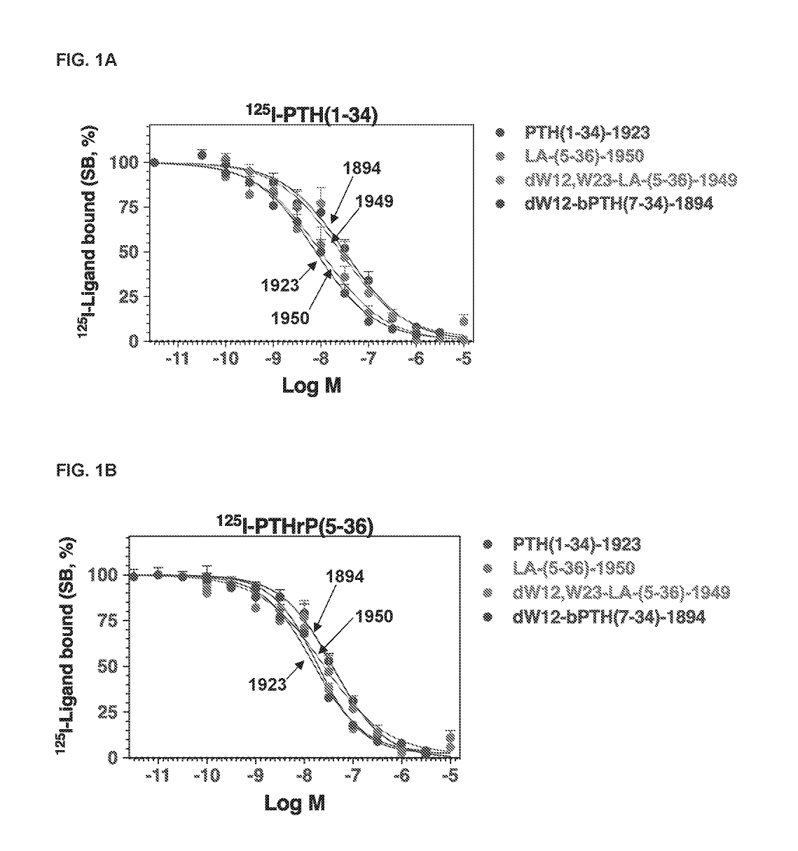

Pharmacological properties of the ligands were assessed in cells expressing the PTHR1 and the GloSensor™ cAMP reporter. The assays used GP-2.3 cells, derived from HEK293 cells by stable transfection with the hPTHR1 and GloSensor™, as well as SGS-72 cells, which are derived from the human osteosarcoma cell line, Saos-2, in which is endogenously expressed the hPTHR1, by stable transfection with GloSensor™. Binding of representative polypeptides to the PTHR1 was assessed in GP-2.3 cells by radioligand competition methods (FIGS. 1A and 1B, Table 3). The polypeptides 1950 and 1949 exhibit apparent PTHR1 affinities that are at least as strong as that for dTrp¹²-bPTH(7-34) analog dTrp¹²-PTH(7-34)-1894.

| | pIC₅₀ | |
|---|---|---|
| | ¹²⁵I-PTH(1-34) | ¹²⁵I-PTHrP(5-36) |
| PTH(1-34)-1923 | 8.18 ± 0.22 | 7.82 ± 0.05 |
| | 6.6 nM | 15.2 nM |
| LA-PTH(5-36)-1950 | 8.05 ± 0.28 | 7.65 ± 0.15 |
| | 9.0 nM | 22.2 nM |
| DW¹², W²³-LA-PTH(5-36)-1949 | 7.65 ± 0.32 | 7.75 ± 0.12 |
| | 22.3 nM | 17.7 nM |
| DW¹², Y³⁴-bPTH(7-34)-1894 | 7.54 ± 0.22 | 7.46 ± 0.08 |
| | 28.6 nM | 35.1 nM |

Table 3 provides results for radioligand competition binding to GP-2.3 (n=4) cells. The assays were performed in GP-2.3 cells with the two tracer ¹²⁵I-radioligands shown; values are half-maximal inhibitory concentration (pIC₅₀) and corresponding nanomolar value (below) derived from curve fitting ligand dose-response data. Data are means of four experiments.

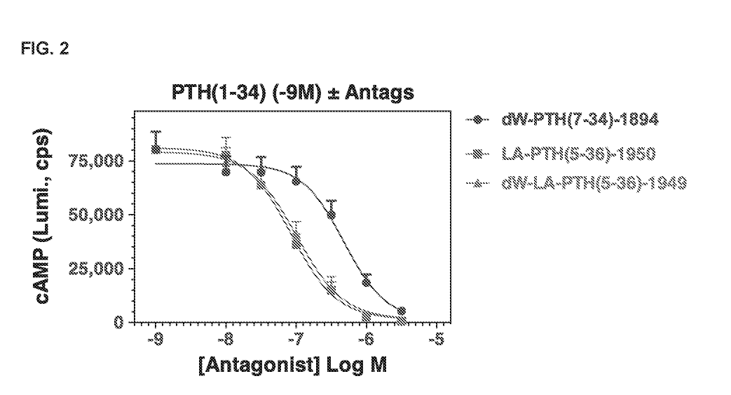

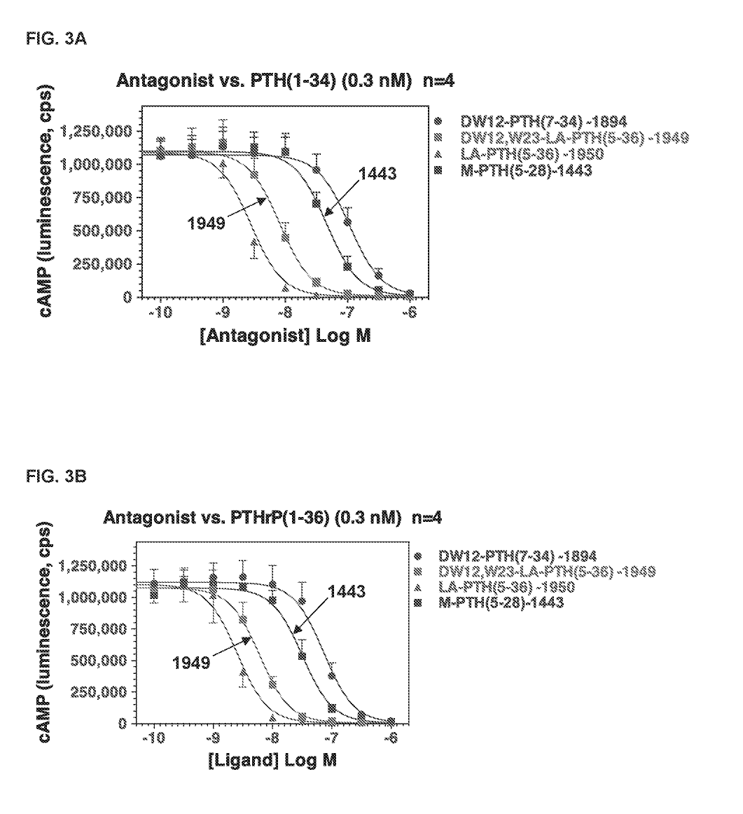

In GP-2.3 cells, LA-PTH(5-36)-1950 inhibited the cAMP signal induced by PTH(1-34) (1 nM) ˜five-fold more effectively than did dTrp¹²-PTH(7-34)-1894 (pIC₅₀s=7.0±0.1 vs. 6.4±0.1 nM), and in SGS-72 cells, it inhibited the cAMP signal induced by PTH(1-34) (0.3 nM) ˜30-fold more effectively (pIC₅₀s=8.6±0.1 vs. 7.2±0.1) (FIGS. 2 and 3A). Similar inhibitory efficacies were observed in SGS-72 cells for PTHrP(1-36) agonist (FIG. 3B).

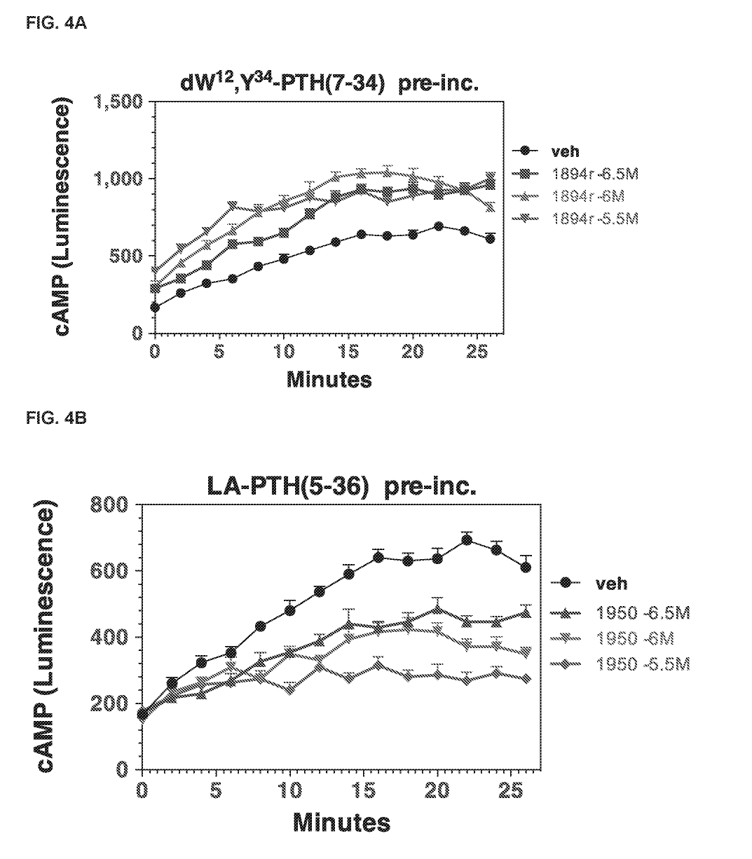

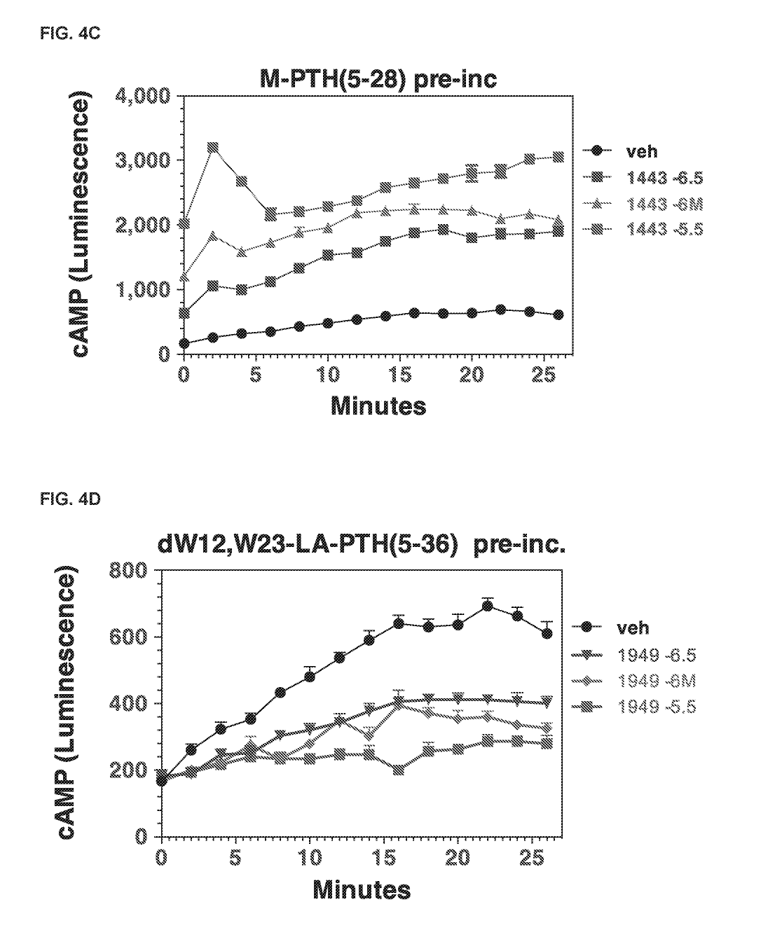

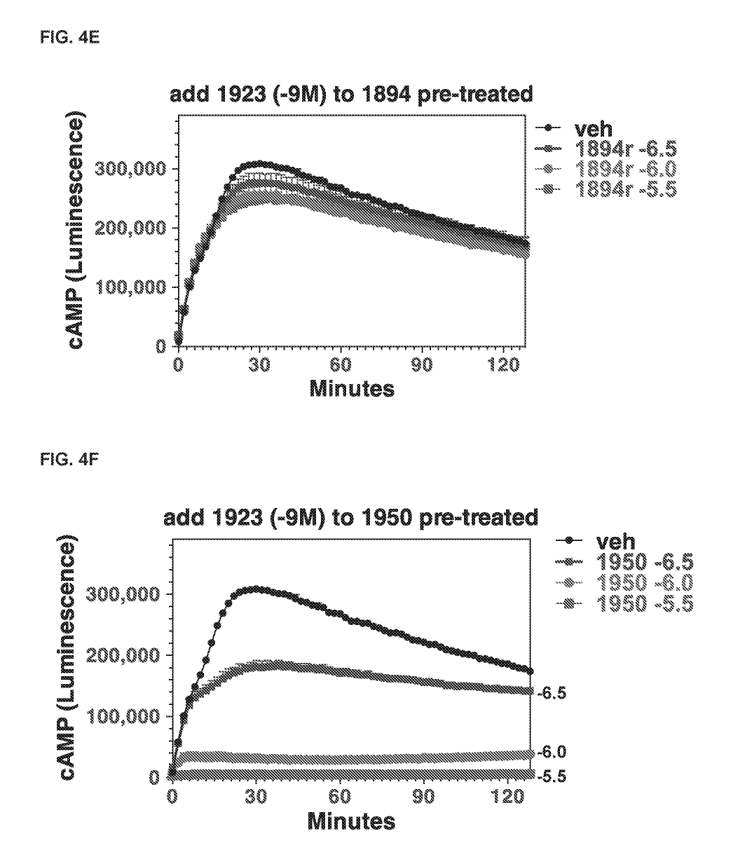

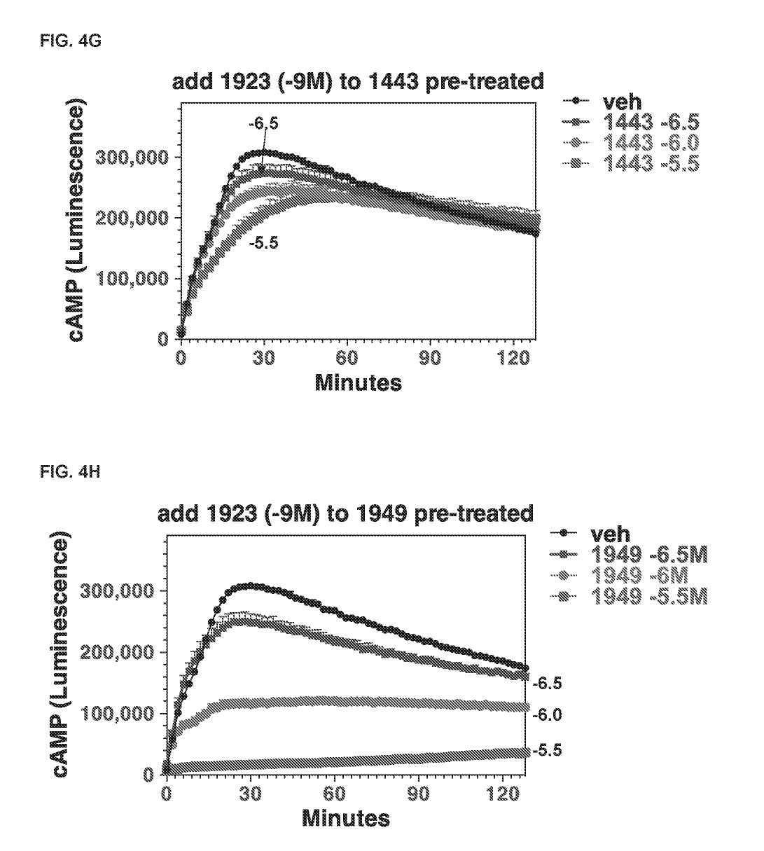

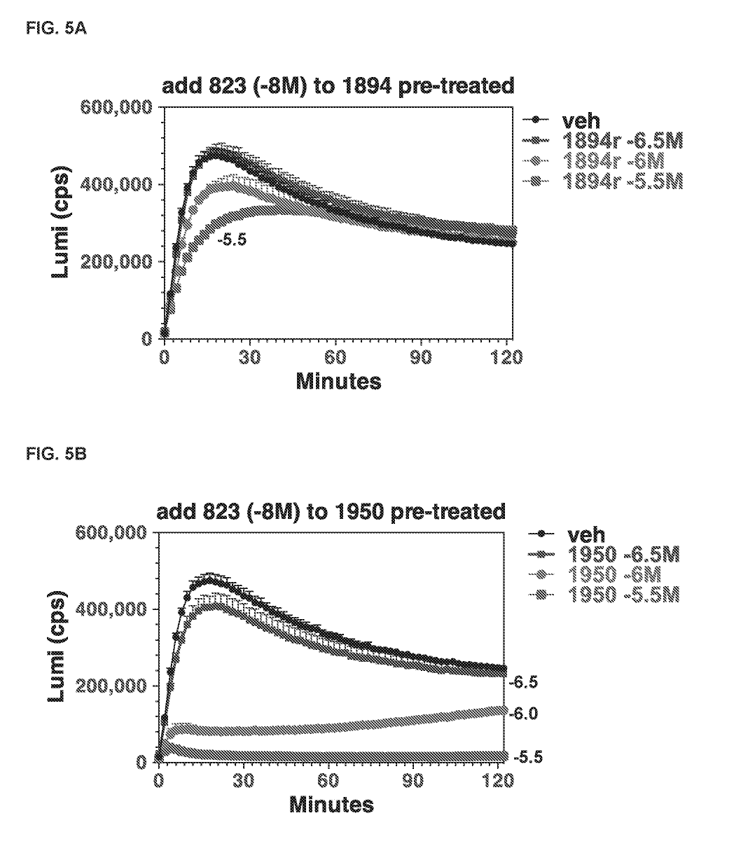

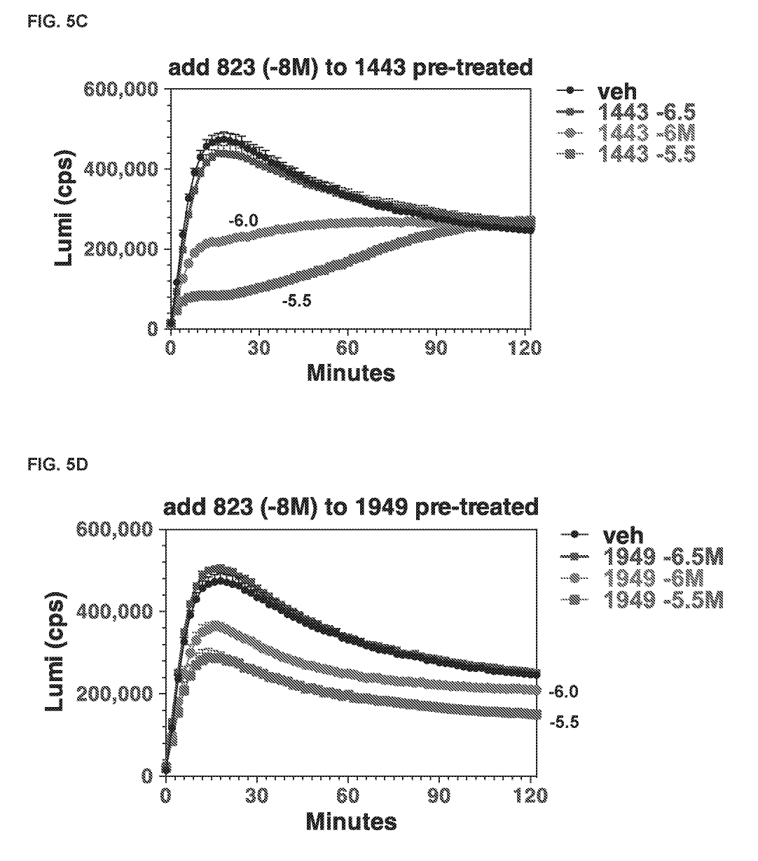

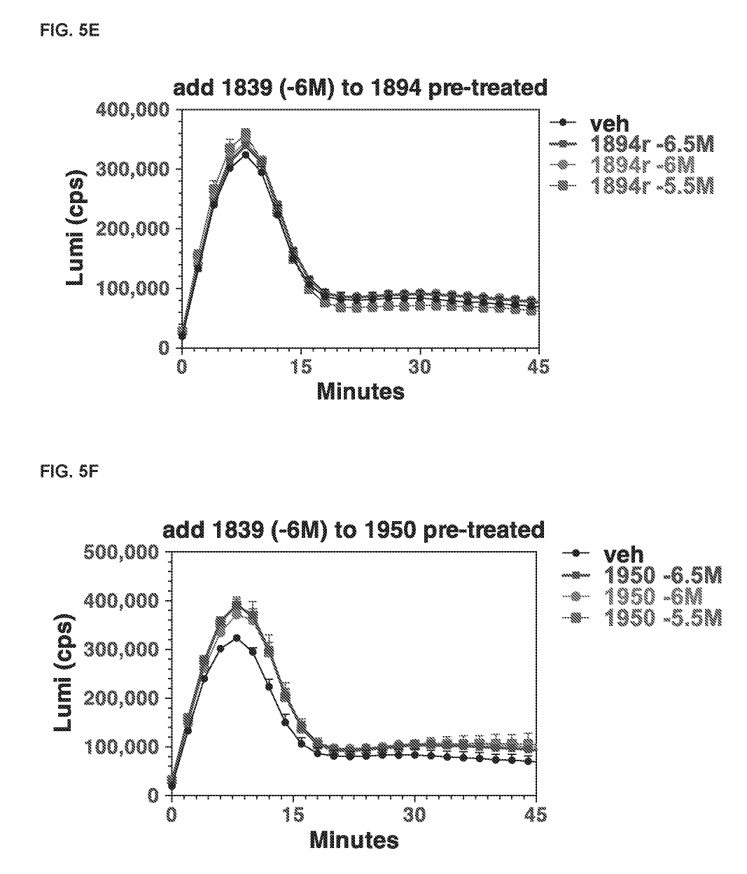

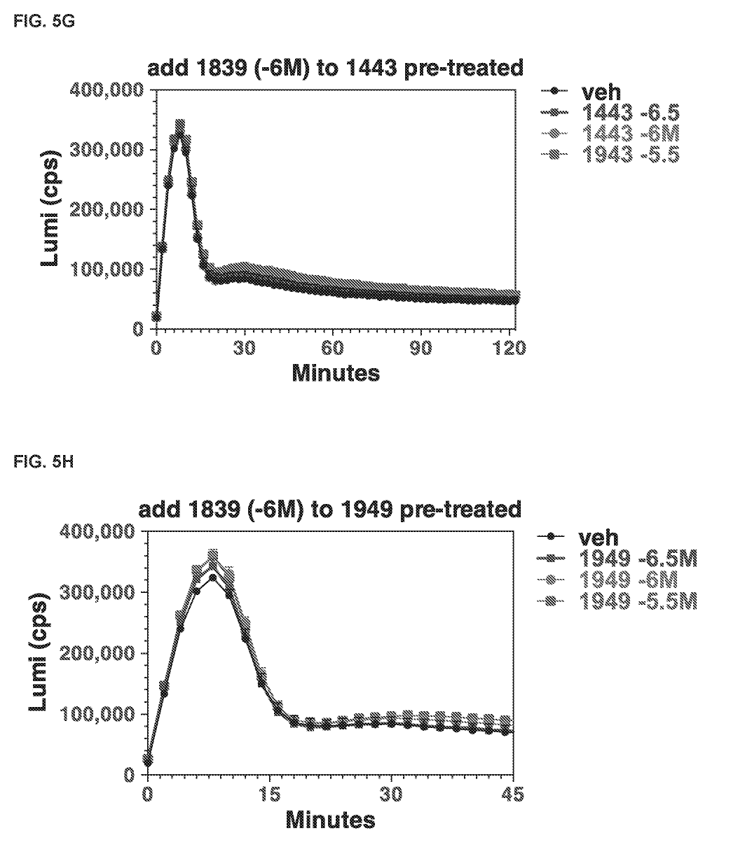

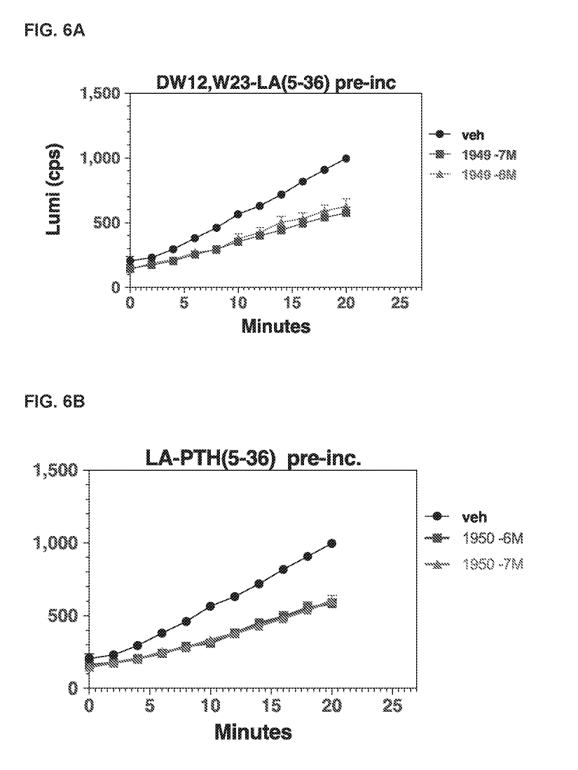

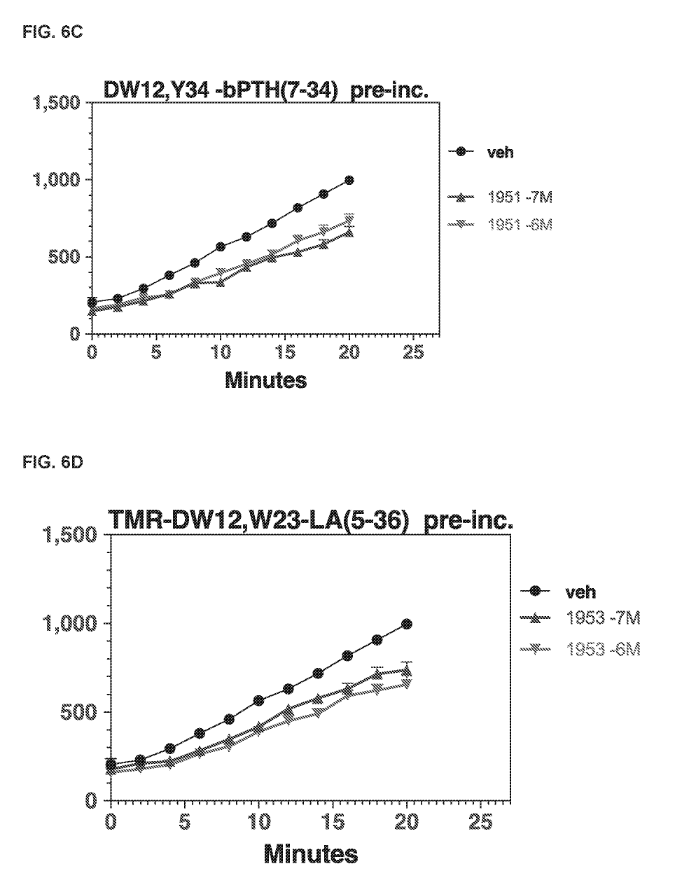

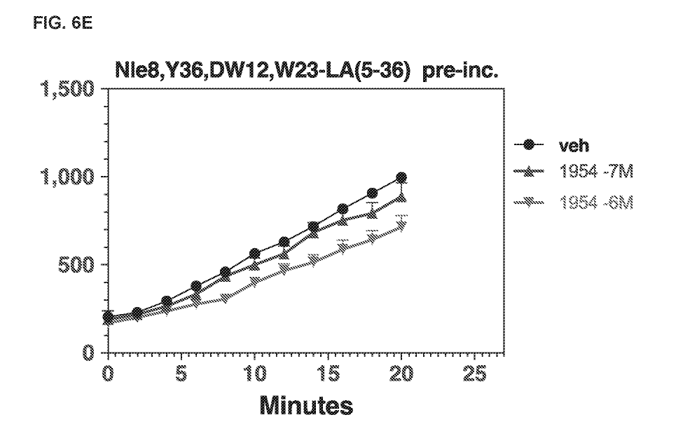

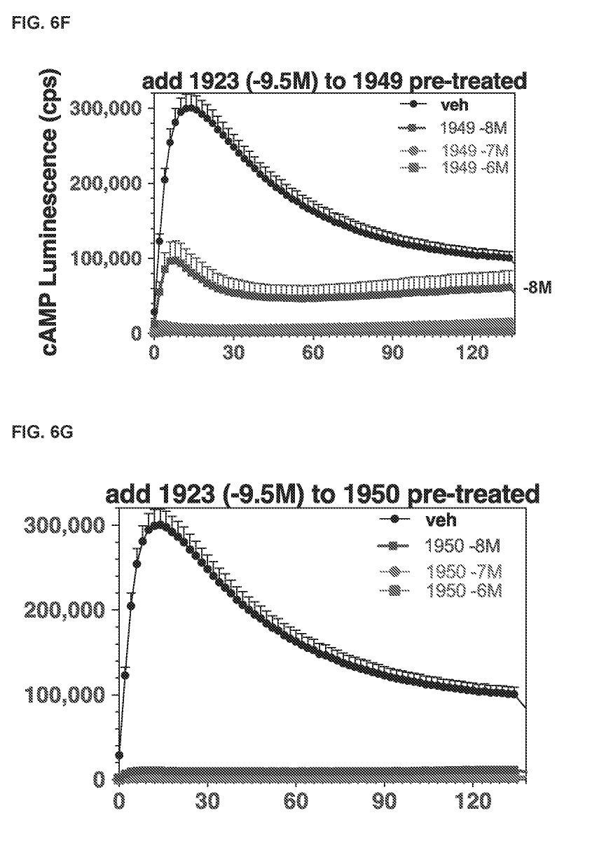

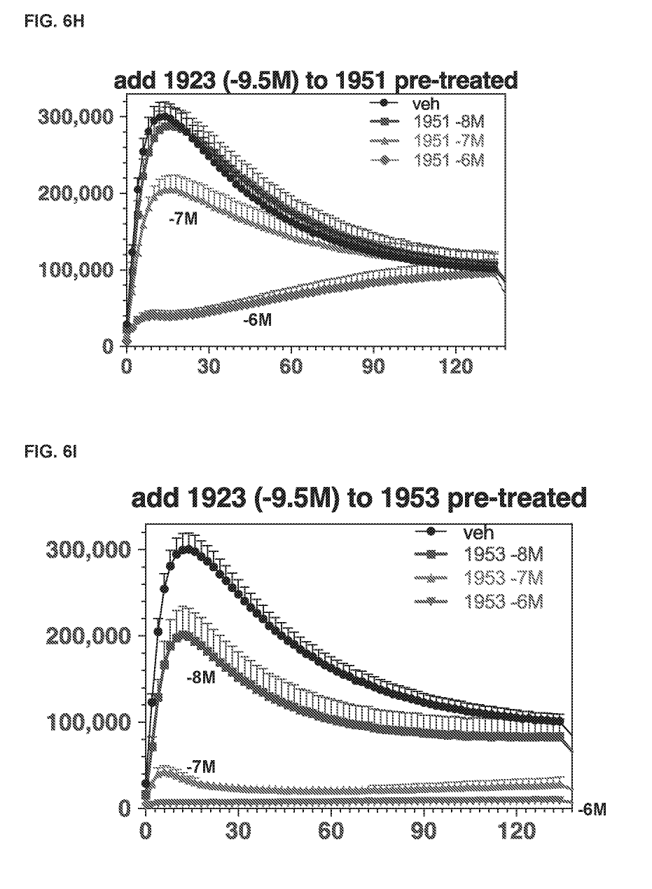

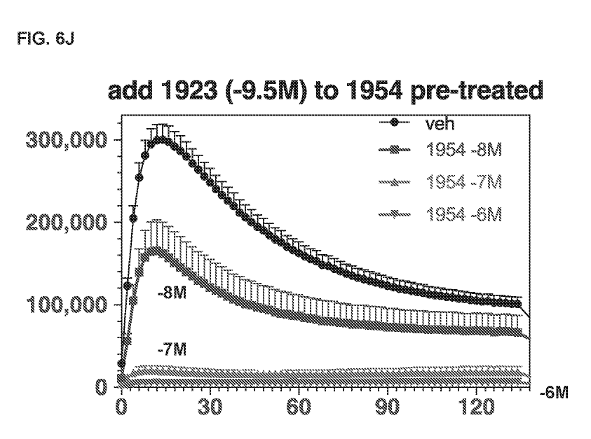

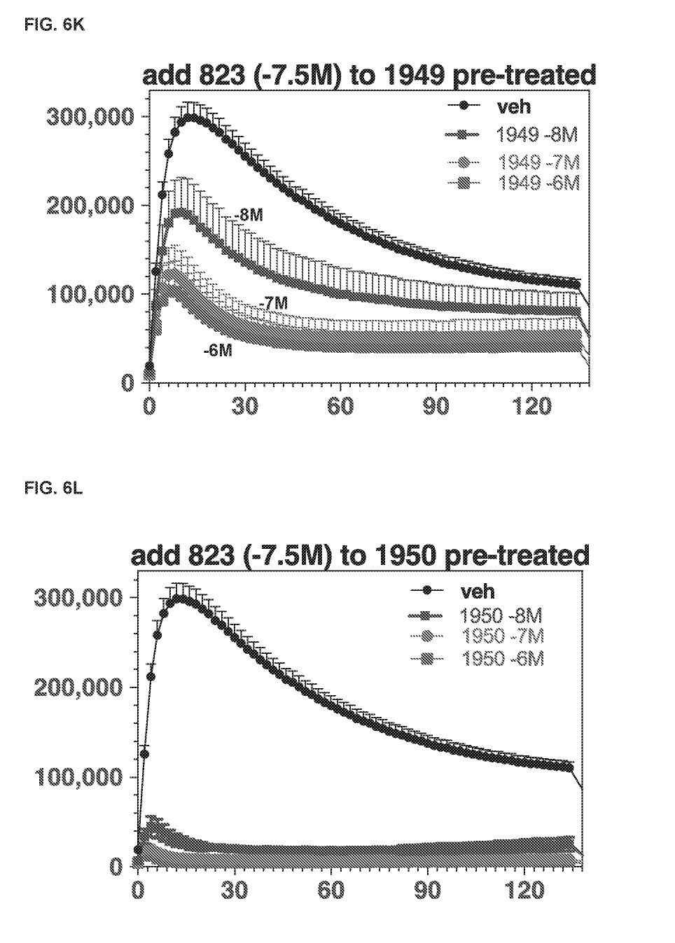

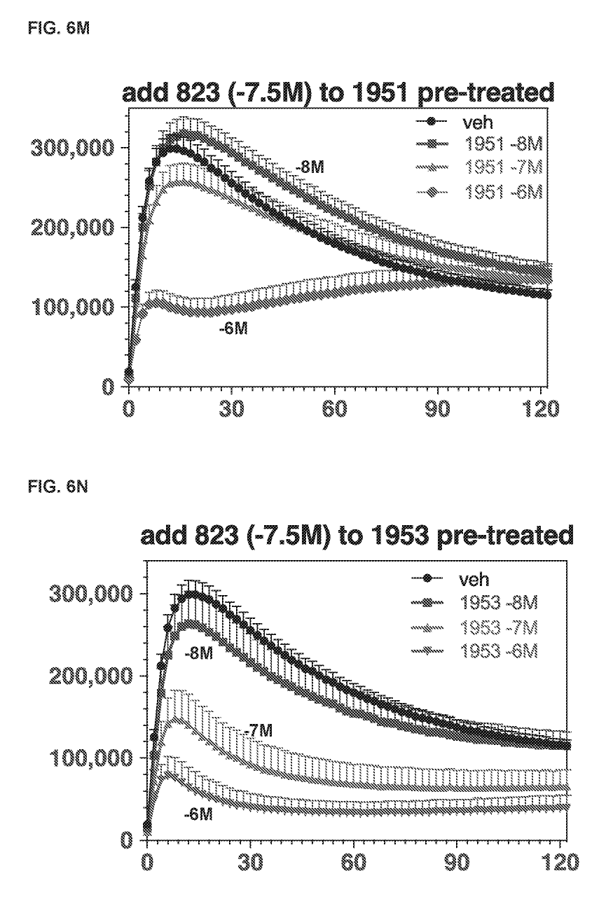

In washout assays, in which GP-2.3 cells were pre-treated with antagonist at varying doses (e.g., 10 μM, 3.3 μM, 1 μM) for 15 minutes and then rinsed three times prior to application of agonist (e.g., PTH(1-34)-1923 at 1.0 nM (GP-2.3), or 0.3 nM (SGS-72), or M-PTH(1-11)-823 10 nM or isoproterenol-1839 1 μM, as a non-specific control that activates endogenous β₂-adrenoreceptors), LA-PTH(5-36)-1950 and dTrp¹², Trp²³-LA-PTH(5-36)-1949 mediated marked (e.g., >90%) and sustained (e.g. >2 hrs) inhibition of the cAMP response induced by the subsequently applied PTHR1 agonist ligand, whereas dTrp¹²-PTH(7-34)-1894 (or the equivalent 1951 used in SGS-72 cell experiments) did not diminish the agonist response, as the cAMP levels reached those seen in the vehicle (Veh) pre-treated cells (FIGS. 4-6). Table 4 summarizes the results for assays of antagonism of PTH(1-34)-induced cAMP response by polypeptides in GP-2.3 cells. Table 5 summarizes the results for assays of antagonism of PTH (1-34)- and PTHrP(1-36)-induced cAMP response in SGS-72 cells.

[symbol]

| Antagonist | pIC50 | P | n |
|---|---|---|---|
| dW12, Y34-bPTH(7-34)-1894 | 6.42 ± 0.05 | 1.000 | 11 |
| | 377 nM | | |
| dW12, W23-LA-PTH(5-36)-1949 | 7.00 ± 0.08 | 0.00034 | 6 |
| | 101 nM | | |
| LA-PTH(5-36)-1950 | 7.22 ± 0.09 | 0.0000025 | 10 |
| | 60 nM | | |
| TMR, dW12, W23-LA-PTH(5-36)- | 6.80 ± 0.08 | 0.0075 | 4 |
| 1953 | 160 nM | | |
| Nle8, dW12, W23, C37-LA-PTH(5- | 6.65 ± 0.13 | 0.18 | 4 |
| 37)-1974 | 224 nM | | |
| TMR, Nle8, dW12, W23, C37-LA- | 5.44 ± 0.06 | 0.0000031 | 4 |
| PTH-(5-37)-1975 | 3610 nM | | |
| TMR, dW12.W23, C37-LA-PTH(5- | 5.71 ± 0.06 | 0.000030 | 4 |
| 37)-1976 | 1961 nM | | |
| TMR, Nle8, dW12, C35-bPTH(7- | 5.55 ± 0.03 | 0.000000003 | 4 |
| 35)-1977 | 2802 nM | | |
| PEG20, TMR, Nle8, dW12, W23, | 6.19 ± 0.13 | 0.16 | 4 |
| C37-LAPTH(5-37)-1978 | 642 nM | | |

[symbol]

indicates data missing or illegible when filed

z,999 concentration of antagonist that inhibited the maximum response to PTH(1-34)-induced in the absence of antagonist (at ˜40 minutes) is reported in units of -log M (pIC50), and the corresponding nanomolar value is shown below each pIC50 value. Data are means of the number of experiments indicated (n). The PTH(1-34)-induced maximum luminescence was ˜80,000 cps and the basal was ˜1,000 cps. TMR indicates Lys13 modified at epsilon amino function with TMR, P vs. inhibition by dW12,Y34-bPTH(7-34).

[symbol]

| | pIC50 | |
|---|---|---|
| Antagonist | vs. PTH(1-34) | vs. PTHrP(1-36) |
| dW12, Y34-bPTH(7-34)-1951 | 8.31 ± 0.13 | 8.33 ± 0.10 |
| | 5 nM | 5 nM |
| dW12, W23-LA-PTH(5-36)-1949 | 9.11 ± 0.13 | 9.10 ± 0.08 |
| | 1 nM | 1 nM |
| LA-PTH(5-36)-1950 | 9.08 ± 0.14 | 9.06 ± 0.11 |
| | 1 nM | 1 nM |
| TMR, Nle8, dW12, W23, C37-LA- | 7.91 ± 0.16 | 8.01 ± 0.10 |
| PTH-(5-37)-1975 | 12 nM | 10 nM |
| PEG20, TMR, Nle8, dW12, W23, | 7.68 ± 0.13 | 7.89 ± 0.08 |
| C37-LAPTH(5-37)-1978 | 21 nM | 13 nM |

[symbol]

indicates data missing or illegible when filed

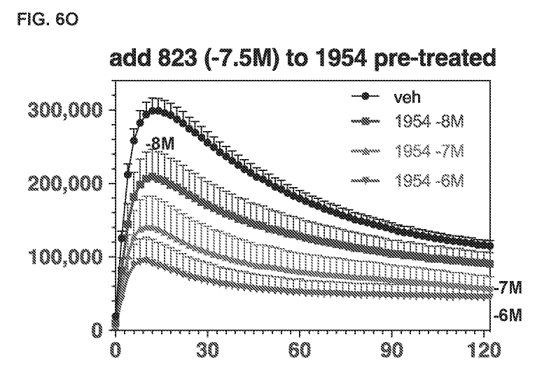

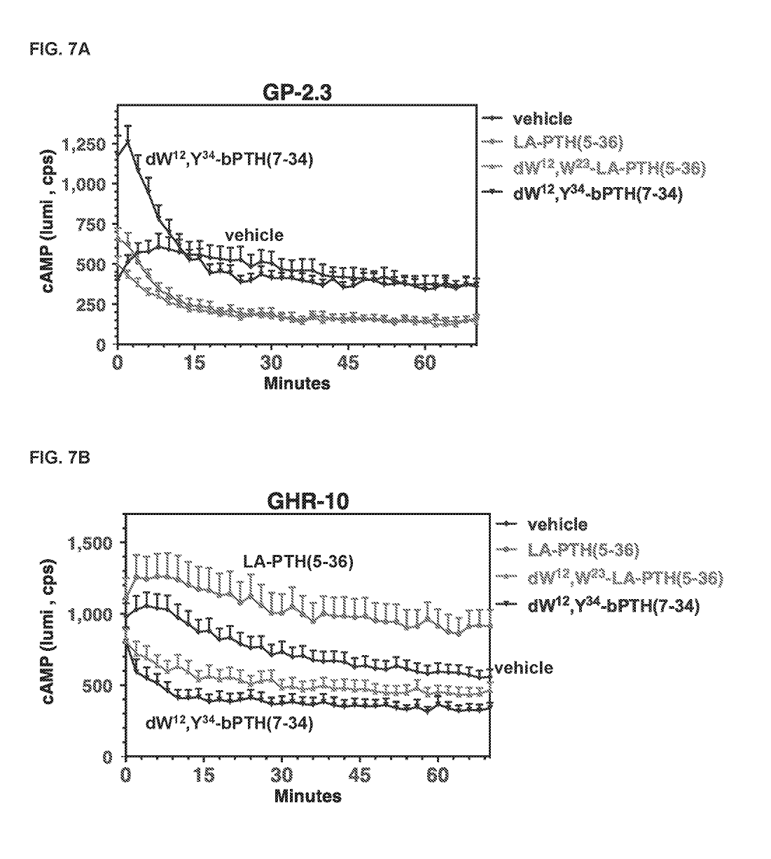

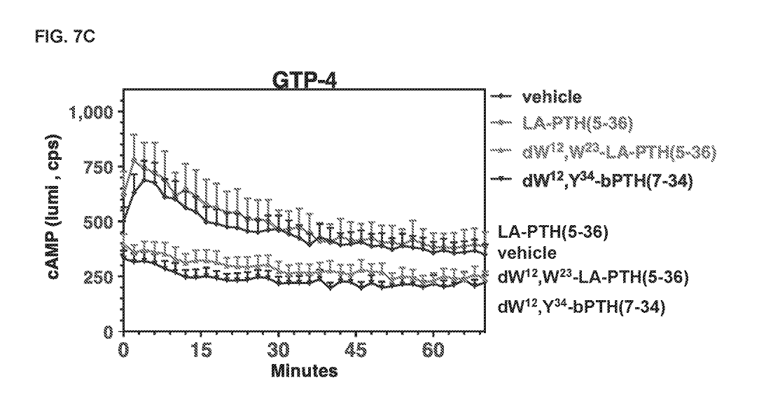

In Table 5, antagonist assays were performed in SGS-72 cells (SaOS2 cells with stable GloSensor™ cAMP reporter) at room temperature. Cells were pre-treated with antagonist at varying concentration for 10 minutes, and then PTH(1-34) or PTHrP(1-36) was added to a concentration of 0.3 nM and peak cAMP-dependent luminescence was recorded and plotted against antagonist ligand concentration. The mean (±sem) concentration of antagonist that inhibited the maximum agonist-induced response observed in the absence of antagonist (at ˜40 minutes) is reported in units of -log M (pIC50), and the corresponding nanomolar value is shown below each pIC50 value. Data are means of the number of seven experiments. The PTH(1-34)-induced maximum luminescence was ˜500,000 cps and the basal was ˜5,000 cps. TMR indicates Lys13 modified at epsilon amino function with TMR. The data are illustrated in FIG. 7A.

Polypeptide dTrp¹², Trp²³-LA-PTH(5-36)-1949, which incorporated the Gly12-->dTrp substitution, exhibited inverse agonist properties on the constitutively active PTHR1 mutants (H223R or T410P) stably expressed in HEK293/GloSensor™ cells (GHR-10 and GTP-4 cell lines, respectively) (FIGS. 7B and 7C). These two PTHR1 mutations are known to cause Jansen's chondrodysplasia.

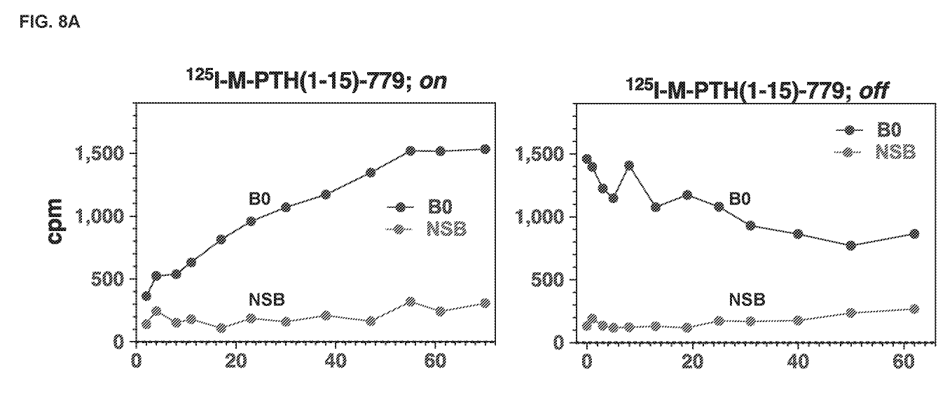

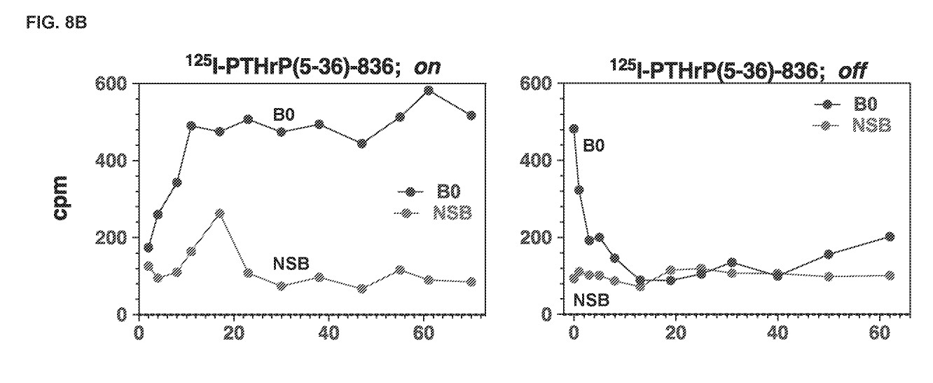

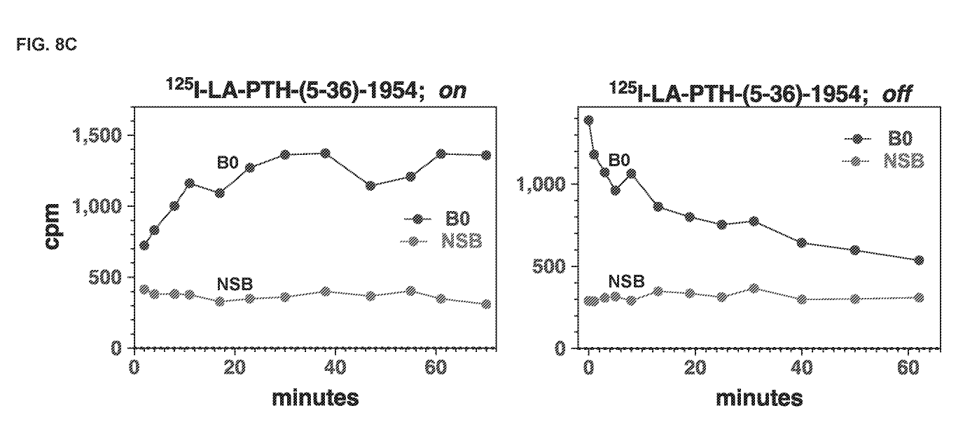

The radioiodinated analog, ¹²⁵I-Nle⁸, dTrp¹², Trp²³, Tyr³⁶-LA-PTH(5-36)-1954, dissociated more slowly from the PTHR1 than did either the conventional antagonist radioligand, ¹²⁵I-Tyr³⁶-PTHrP(5-36)-836, or the N-terminal agonist radioligand ¹²⁵I-M-PTH(1-15)-779, which corresponds to ¹²⁵I-Aib^{1,3}, Gln¹⁰, hArg¹¹, Ala¹², Trp¹⁴-PTH(1-15) (FIGS. 8A and 8B).

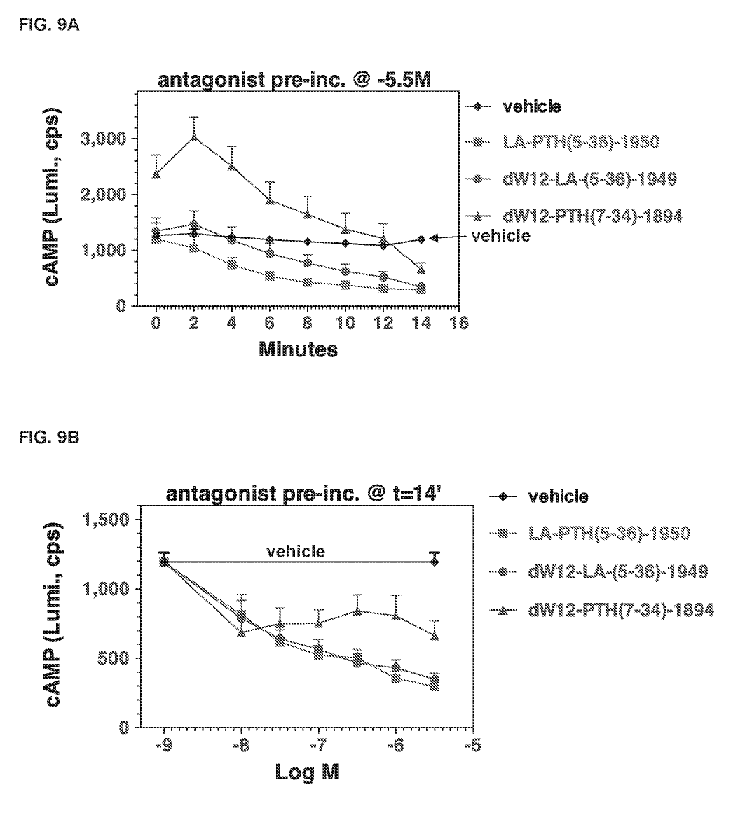

Polypeptides dTrp¹², Trp²³-LA-PTH(5-36)-1949 and LA-PTH(5-36)-1950 exhibited less residual agonist activity than did the conventional antagonist, dTrp¹²-PTH(7-34)-1894, as assessed by applying these ligands directly to GP-2.3 cells and assessing cAMP-dependent luminescent responses from GloSensor™ reporter (FIGS. 9A and 9B).

Incorporation of tetramethylrhodamine (TMR) at the epsilon amino side chain atom of Lys¹³ conferred fluorescent properties to LA-PTH antagonist polypeptide, and thus enabled visual demonstration that TMR-LA-PTH(5-36)-1953 effectively retained bound PTHR1 on the cell surface, and thus did not internalize into the cell cytoplasm of GP-2.3 cells at 15 minutes after ligand addition (100 nM), whereas the agonist, TMR-PTH(1-35)-1962 internalized into endosomal vesicles (FIGS. 10A and 10B).

Intravenous co-injection of dTrp¹², Trp²³-LA-PTH(5-36)-1949 into mice with PTH(1-34) inhibited the capacity of the injected PTH(1-34) agonist to induce a calcemic response (FIG. 11), thus confirming that the LA-PTH(5-36) scaffold can be used as an effective PTHR1 antagonist in vivo.

### In Vitro Antagonism at Endogenous PTHR1 in SGS-72 Cells

Antagonism at the endogenous hPTHR1 was assessed in SGS-72 cells; cells were pretreated with vehicle or vehicle containing varying doses of antagonist ligand for 15 minutes, then PTH(1-34) or PTHrP(1-36) was added at a concentration of 0.3 nM, and GloSensor™-cAMP responses were measured. The results are summarized in Table 6. IC50s indicate that antagonist concentration at which the response maximum observed in vehicle treated cells was inhibited by 50%. Date are means (±SEM) of the number of separate experiments indicated by n.

TABLE 6

| Ref | | | pIC50 | | |
|---|---|---|---|---|---|
| # | Chemical Name | PTH(1-34) | n | PTHrP(1-36) | n |
| 1894 | dW12, Y-bPTH(7-34)- | 7.84 ± 0.15 | | | |
| | 1894/1951 | 14.5 nM | | | |
| 1949 | dW12, W23-LA-PTH(7-36)- | 8.84 ± 0.12 | 18 | 8.92 ± 0.19 | 12 |
| | 1949 | 1.45 nM | | 1.21 nM | |
| 1950 | LA-PTH(5-36)-1950 | 9.21 ± 0.11 | 23 | 9.02 ± 0.14 | 12 |
| | | 0.61 nM | | 0.96 nM | |
| 1953 | TMR-dW12, W23-LA-PTH(5- | 8.49 ± 0.11 | 11 | | |
| | 36)-1953 | 3.23 nM | | | |
| 1954 | Nle8, Y36, dW12, W23-LA- | 8.76 ± 0.14 | 7 | | |
| | PTH(5-36)-1954 | 1.75 nM | | | |
| 1974 | Nle8, dW12, W23, C37-LA- | 8.36 ± 0.15 | 4 | | |
| | PTH(5-37)-1974 | 4.39 nM | | | |
| 1975 | Nle8, TMR, dW12. W23, C37- | 7.69 ± 0.13 | 18 | 8.01 ± 0.10 | 7 |
| | LA(5-37)-1975 | 20.32 nM | | 9.71 nM | |
| 1976 | TMR-dW12. W23, C37. LA-(5- | 7.73 ± 0.15 | 11 | | |
| | 37)-1976 | 18.50 nM | | | |
| 1978 | PEG20- | 7.62 ± 0.09 | 11 | 7.89 ± 0.08 | 7 |
| | Nle8. TMR, dW12. W23, C37- | 24.14 nM | | 12.92 nM | |
| | LA(5-37)-1978 | | | | |
| 1990 | dW12, W23-LA-PTH(7-36)- | 8.91 ± 0.07 | 5 | | |
| | 1990 | 1.23 nM | | | |
| 1991 | TMR-dW12, W23-LA-PTH(7- | 8.71 ± 0.14 | 4 | | |
| | 36)-1991 | 1.95 nM | | | |
| 1992 | Nle, dW12, W23-LA-PTH(7- | 8.81 ± 0.07 | 5 | | |
| | 36)-1992 | 1.55 nM | | | |
| 1993 | TMR-Nle, -dW12, W23-LA- | 8.48 ± 0.08 | 5 | | |
| | PTH(7-36)-1993 | 3.34 nM | | | |
| 1997 | Nle8, L11, dW12, W23-LA- | 8.68 ± 0.09 | 5 | | |
| | PTH(7-36)-1997 | 2.10 nM | | | |
| 1998 | TMR-Nle8, L11, dW12, W23- | 8.65 ± 0.07 | 4 | | |
| | LA-PTH(7-36)-1998 | 2.21 nM | | | |
| 1999 | Nle8, L11, dW12, W23-LA- | 8.56 ± 0.12 | 5 | | |
| | PTH(5-36)-1999 | 2.75 nM | | | |
| 2000 | TMR-Nle8, L11, dW12, W23- | 8.52 ± 0.14 | 4 | | |
| | LA-PTH(5-36)-2000 | 3.02 nM | | | |
| 2001 | L11, dW12, W23-LA-PTH(7- | 8.68 ± 0.06 | 5 | | |
| | 36)-2001 | 2.08 nM | | | |
| 2002 | TMR-L11, dW12, W23-LA- | 8.83 ± 0.09 | 4 | | |
| | PTH(7-36)-2002 | 1.49 nM | | | |
| 2003 | L11, dW12, W23-LA-PTH(5- | 8.59 ± 0.18 | 5 | | |
| | 36)-2003 | 2.57 nM | | | |
| 2004 | TMR-L11, dW12, W23-LA- | 8.67 ± 0.08 | 4 | | |
| | PTH(5-36) | 2.13 nM | | | |

### Effect of PTHR1 Antagonists on Blood Ca²⁺ Levels in Wild-Type Mice

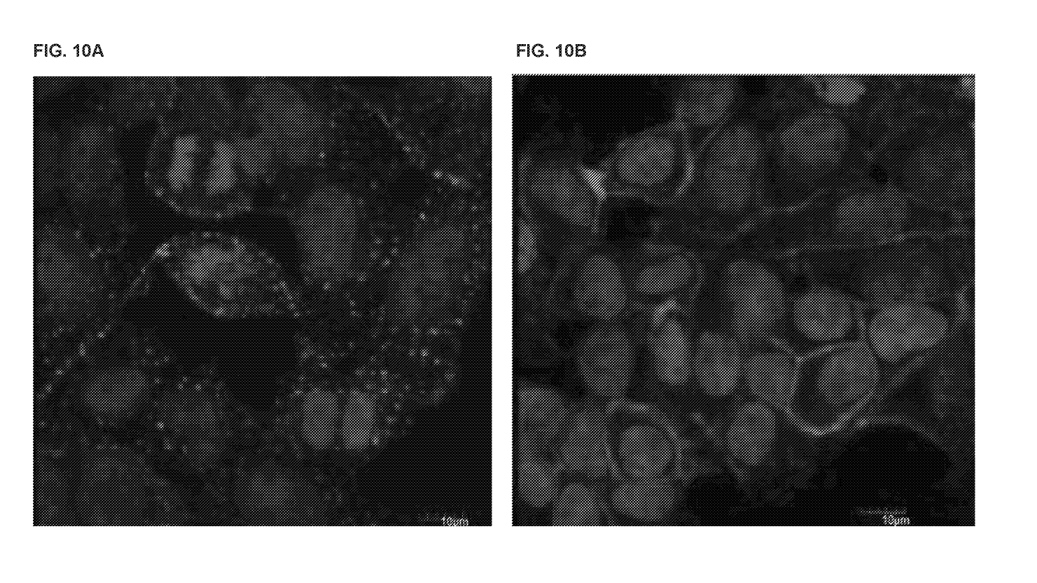

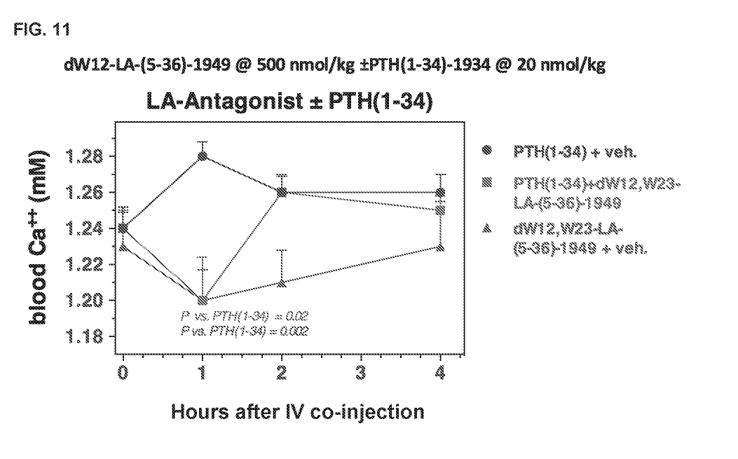

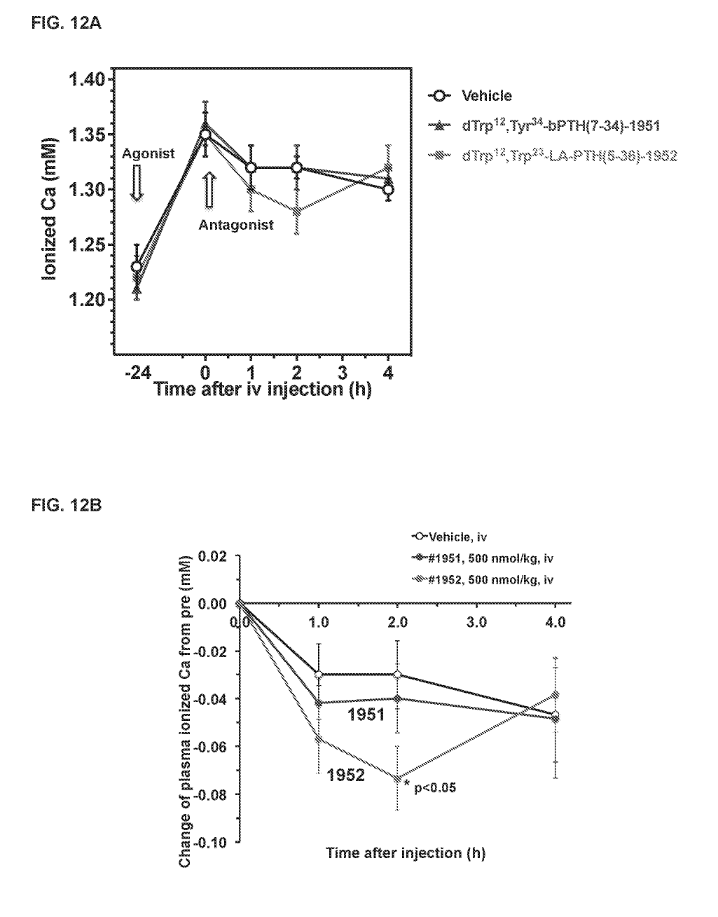

To study antagonism at the wild-type PTHR under conditions of excess PTH, as occurs in humans with hyperparathyroidism, we developed a mouse non-genetic, transient model of hyperparathyroidism. Wild-type mice were injected with a PTH agonist analog, PEG-PTH(1-35), which induces hypercalcemia lasting between 24 and 72 hours after a single s.c. injection (see Guo et al., Journal of Bone and Mineral Research, 32:86-98, 2017). Antagonists were injected after 24 hours following the injection of PEG-PTH(1-35) and effects on blood Ca²⁺ levels were measured. The results are illustrated in FIGS. 12A and 12B.

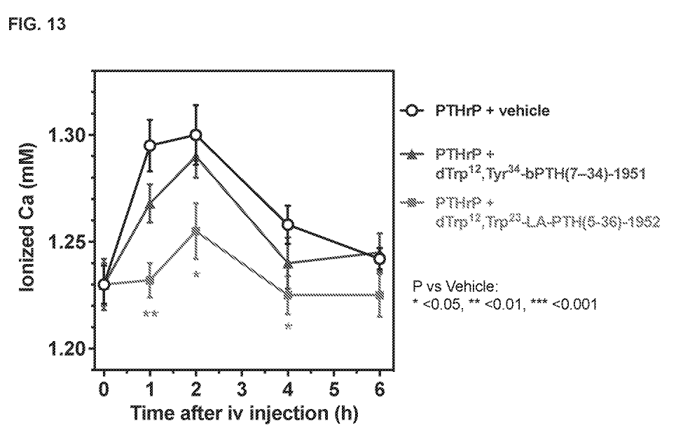

To study antagonism at the wild-type PTHR under conditions of excess PTHrP, as occurs in humans with hypercalcemia of malignancy, we co-injected wild-type mice with PTHrP(1-36) and a test antagonist and measured effects on blood Ca²⁺ levels. The results are illustrated in FIG. 13.

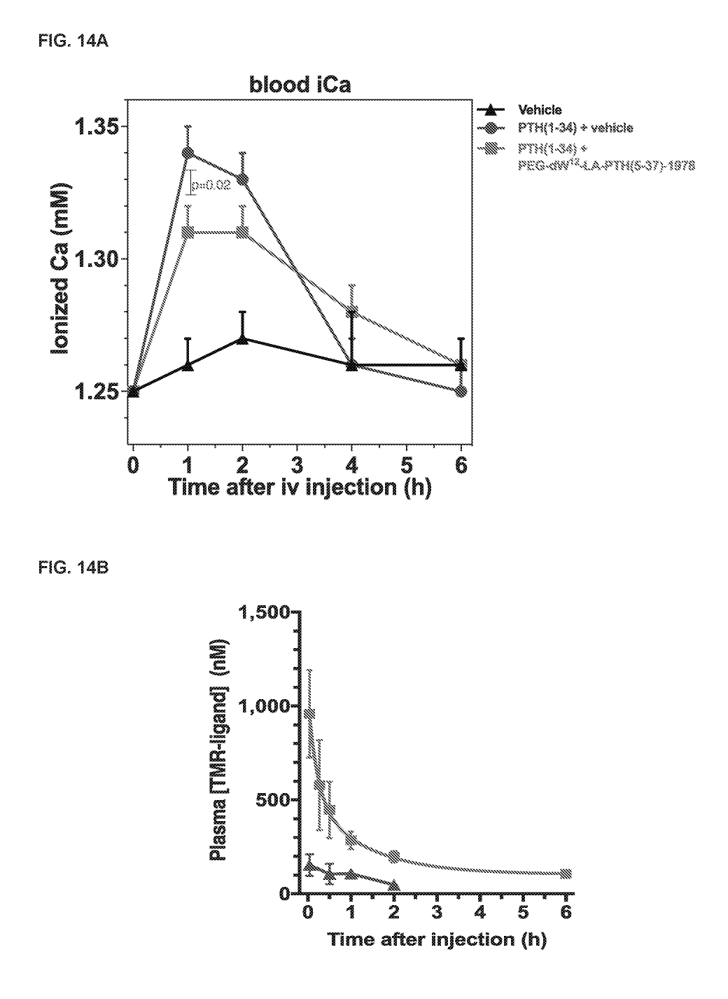

PEG-LA-PTH(5-37)-1978 was tested for antagonism in wild-type mice by co-injection with PTH(1-34). The observed blood Ca²⁺ levels demonstrated antagonist effect of PEG-LA-PTH(5-37)-1978 (see FIG. 14A).

### Pharmacokinetic Effect of PEG Substitution

The pharmacokinetic effect of pegylation on the antagonist/inverse agonist peptides was assessed by comparing PEG-LA-PTH(5-37)-1978 plasma levels to LA-PTH(5-37)-1975 plasma levels (for an example of similar tests, see Guo et al., Journal of Bone and Mineral Research, 32:86-98, 2017). Mice (9-week old female CD1 strain) were injected with PEG-LA-PTH(5-37)-1978 (30 nmol/kg) or LA-PTH(5-37)-1975 (30 nmol/kg), and TMR fluorescence in plasma was measured. The TMR fluorescence signal was converted to ligand concentration (nM) using a standard curve generated with the same corresponding ligand (Guo et al., Journal of Bone and Mineral Research, 32:86-98, 2017). Data are means±SE; n=3. t_{1/2}=0.9 h for PEG-LA-PTH(5-37)-1978, t_{1/2} was not determined for LA-PTH(5-37)-1975. The results are illustrated in FIG. 14B.

### Inverse Agonist Properties

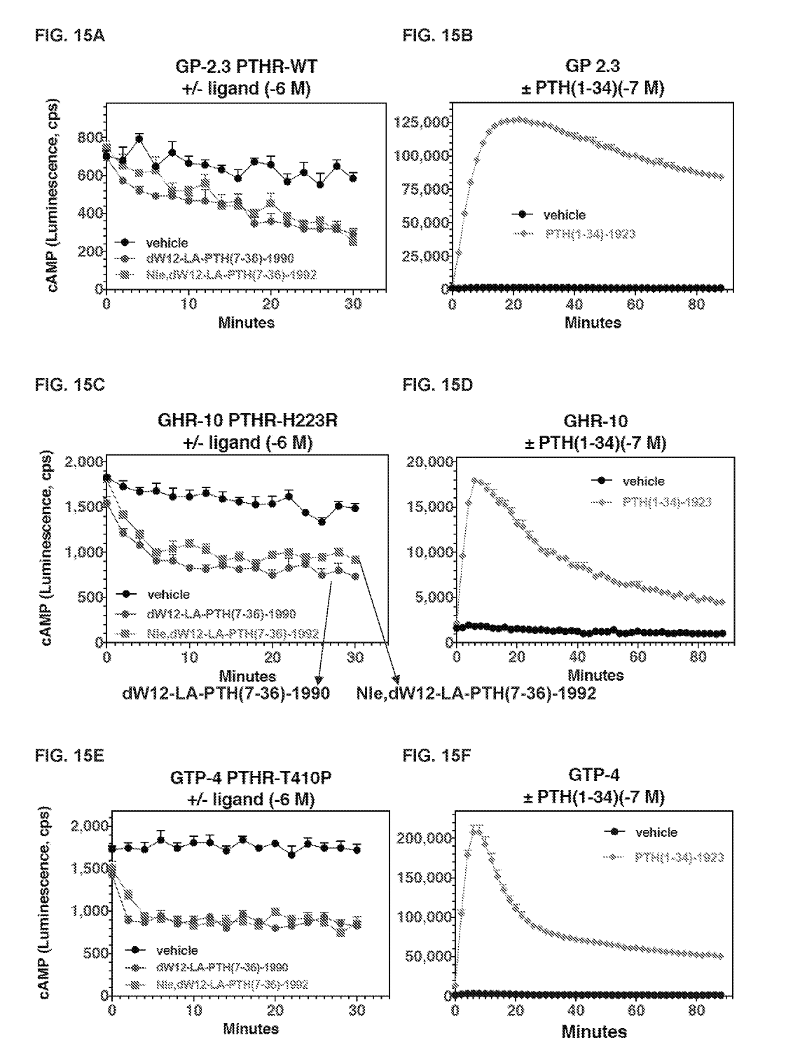

These experiments were performed in HEK-293 cells stably transfected to express the GloSensor™ cAMP reporter along with a constitutively active mutant human PTHR1. The HEK-293 cells stably expressing GloSensor™ cAMP reporter along with a constitutively active mutant human PTHR1-H223R are GHR-10 cells. The HEK-293 cells stably expressing GloSensor™ cAMP reporter along with a constitutively active mutant human PTHR1-T410P are GTP-4 cells. The HEK-293 cells stably expressing GloSensor™ cAMP reporter along with a WT-PTHR1 are GP-2.3 cells. GP-2.3 cells were used as control. Cells in 96-well plates were treated with either vehicle or vehicle containing inverse agonist peptide dW12,W23-LA-PTH(7-36)-1990 or Nle8, dW12, W23-LA-PTH(7-36)-1992, or with agonist PTH(1-34)-1923. Effects on basal cAMP signaling over time were assessed during periods of direct ligand addition (FIGS. 15A-15F). The measurements were performed by recording GloSensor™ luminescence as counts per second (cps) using a PerkinElmer Envision plate reader. The data are illustrated in FIGS. 15A-15F. Data are from triplicate wells (means±SEM) of a single experiment representative of two others. The reduction in signal, relative to vehicle, induced by ligands 1990 or 1992 indicates an inverse agonist response.

Persistence of the antagonist/inverse agonist effects (prolonged action) as follows. Effects of eight peptides on basal and PTH(1-34)-stimulated cAMP signaling by PTHR-H223R were assessed in GHR-10 cells (HEK-293-derived cells stably expressing glosensor cAMP reporter and PTHR1-H223R). Time courses of changes in cAMP levels were recorded in three consecutive phases:

1) after initial addition of a candidate antagonist/inverse agonist (1×10⁻⁶ M-1×10⁻⁹ M) to cells pre-loaded for −30 minutes with luciferin (left column);

2) after removal of unbound inverse agonist by extensive wash-out with buffer and replenishment with fresh luciferin-containing buffer (middle column); and

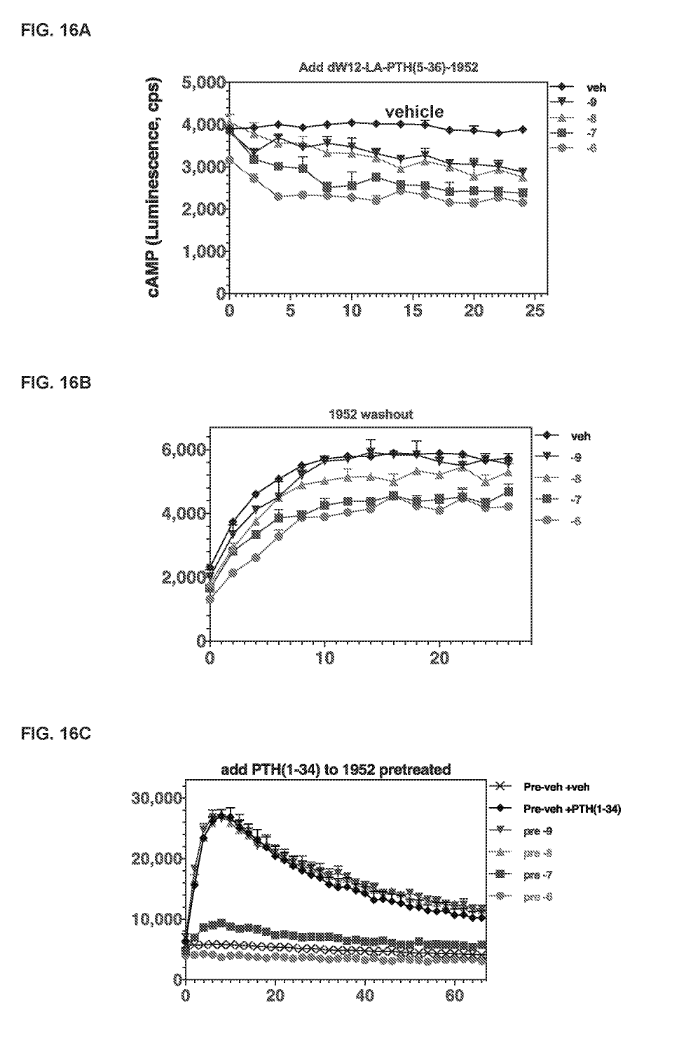

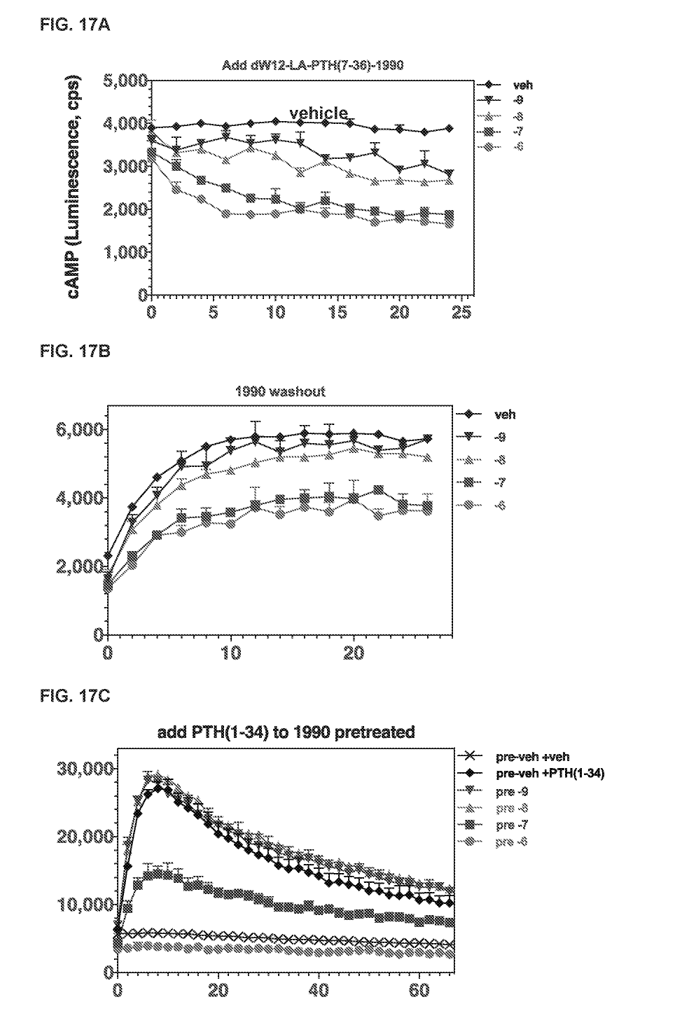

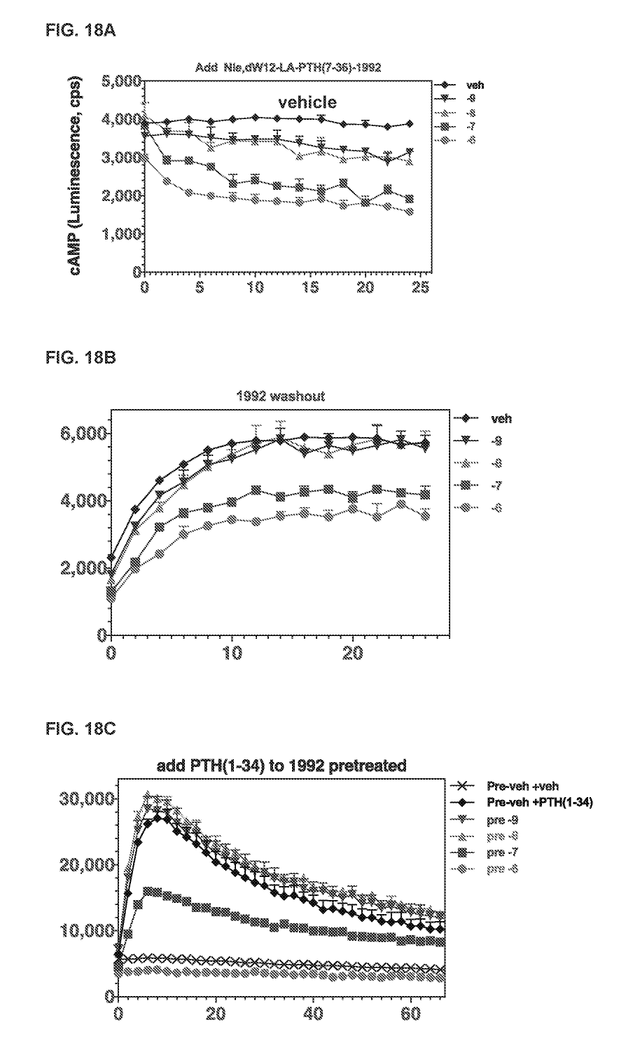

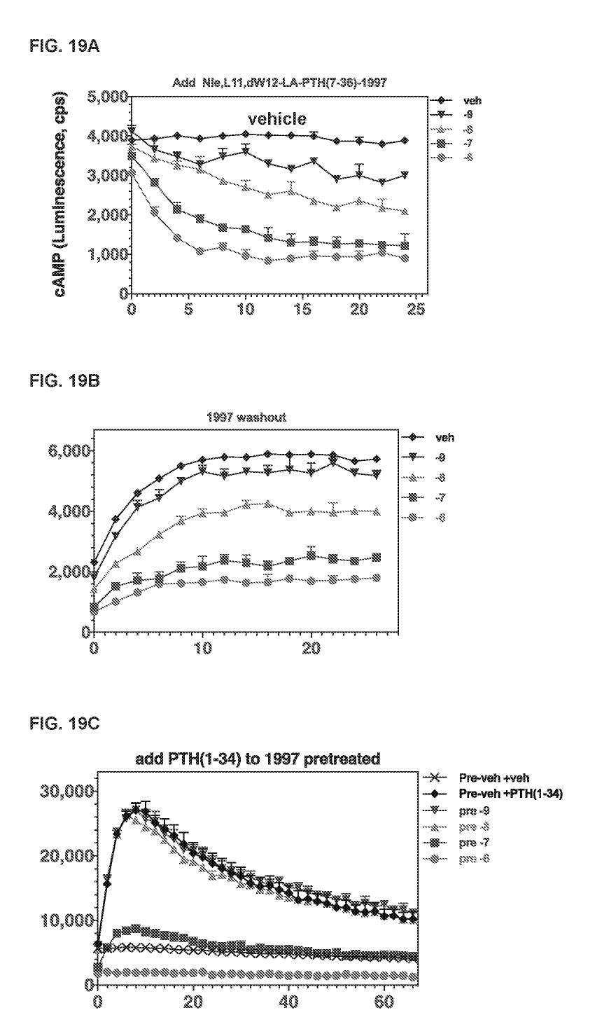

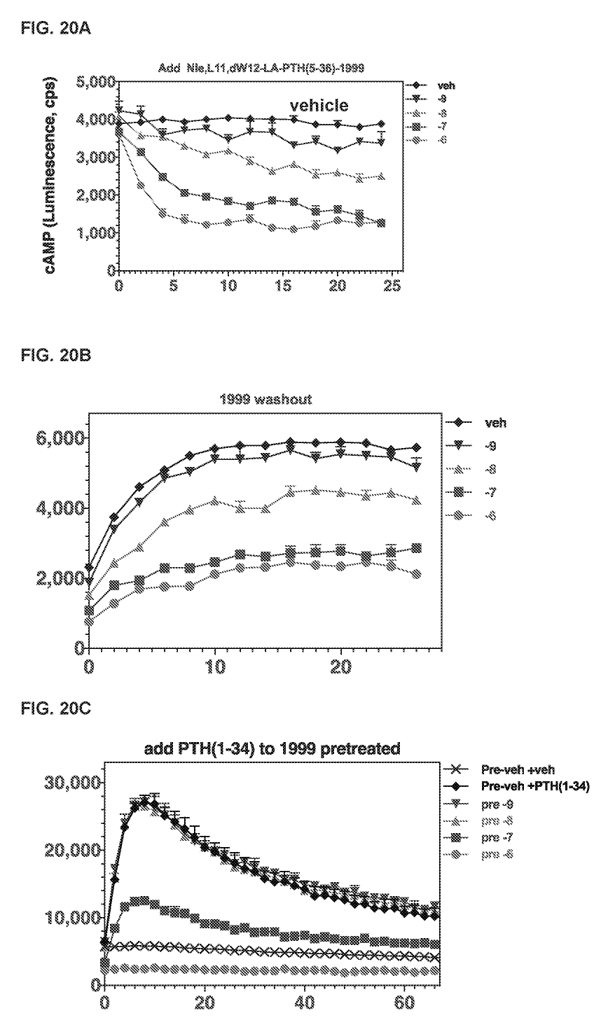

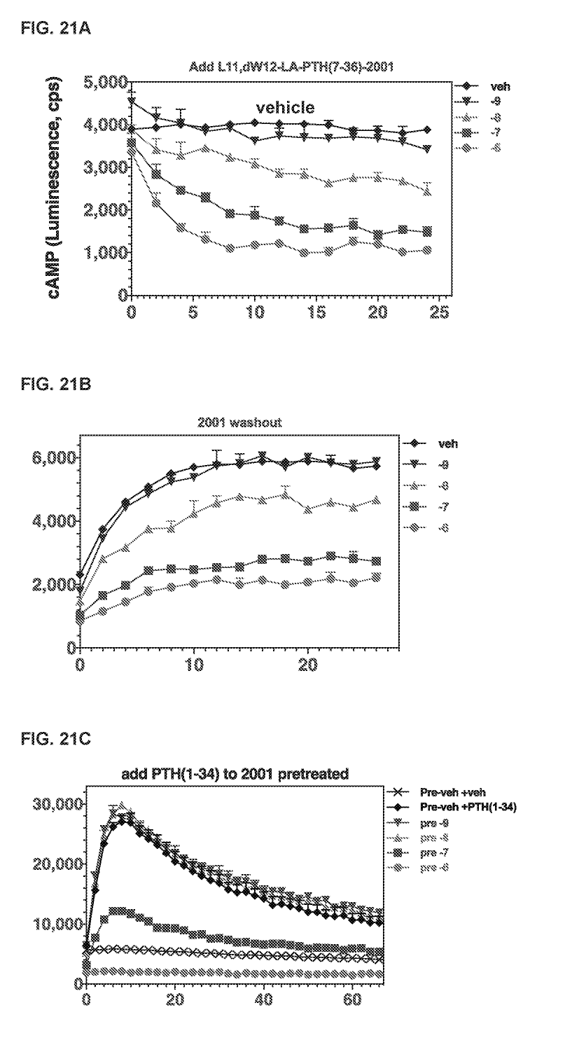

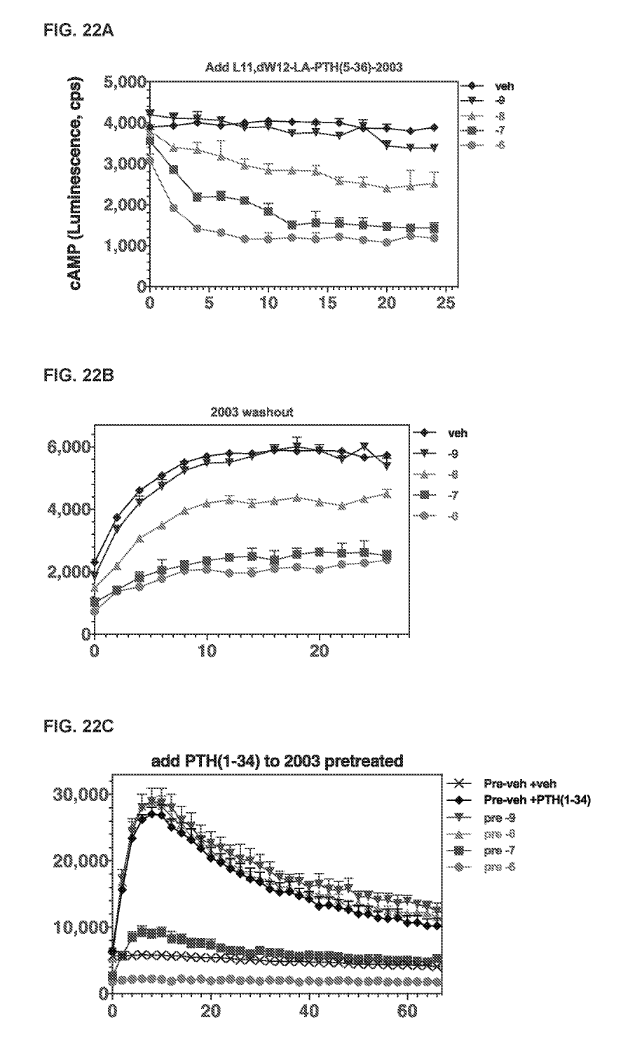

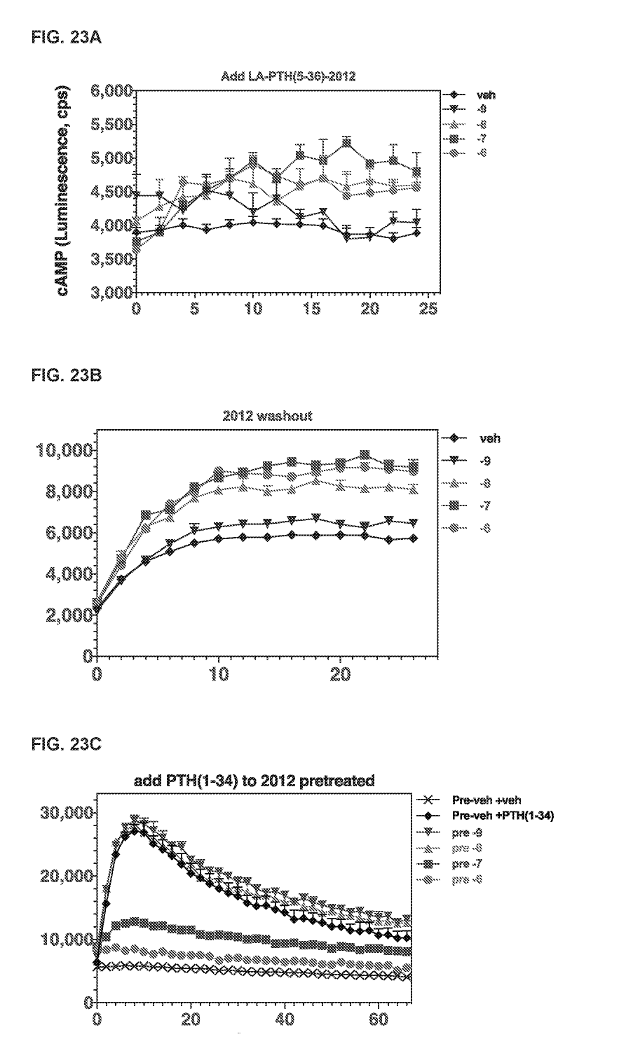

3) after addition of PTH(1-34) (3 nM) to the washed-out cells (right column). Control wells were treated with vehicle. GloSensor™ luminescence, as counts per second (cps), was recorded using a PerkinElmer Envision plate reader. Data are from duplicate wells (means±SEM) of a single experiment representative of two others. The results are illustrated in Table 7 and in FIGS. 16A-16C, 17A-17C, 18A-18C, 19A-19C, 20A-20C, 21A-21C, 22A-22C, and 23A-23C. The reduced signal, relative to vehicle, in the washout phase with certain ligands indicates persistence of inverse agonist response, and the reduction in the response to added PTH(1-34) (compare to Pre-vehicle+PTH(1-34) traces) indicates persistent antagonism by receptor occupancy of the candidate inverse agonist.

TABLE 7

| | | Reduction of basal | |
|---|---|---|---|
| | | cAMP, % vehicle | |
| | | | After |
| | | Ligand | ligand |
| ref. No. | Chemical Name | @ −6M | washout |
| 1952 | dW12, W23-LA-PTH(5-36)-1952 | 41 | 30 |
| 1990 | dW12, W23-LA-PTH(7-36)-1990 | 50 | 39 |
| 1991 | TMR-dW12, W23-LA-PTH(7-36)-1991 | | |
| 1992 | Nle, dW12, W23-LA-PTH(7-36)-1992 | 51 | 40 |
| 1997 | Nle8, L11, dW12, W23-LA-PTH(7-36)- | 70 | 71 |
| | 1997 | | |
| 1999 | Nle8, L11, dW12, W23-LA-PTH(5-36)- | 63 | 62 |
| | 1999 | | |
| 2001 | L11, dW12, W23-LA-PTH(7-36)-2001 | 66 | 65 |
| 2003 | L11, dW12, W23-LA-PTH(5-36)-2003 | 66 | 64 |
| 2004 | TMR-L11, dW12, W23-LA-PTH(5-36)- | | |
| | 2004 | | |
| 2012 | LA-PTH(5-36)-2012 | −15 | −49 |

The data in Table 7 were obtained as follows. GHR10 cells were treated with vehicle or vehicle containing the indicated peptide for 24 minutes and cAMP-dependent luminescence was recorded at two-minute intervals. The cells were then washed to remove unbound ligand and media containing fresh luciferin was added, and cAMP luminescence was recorded for another 26 minutes. Percentage reduction was calculated from the area-under-the-curve (AUC) values derived from the time course plots using the equation:

$$100 \times ( 1 - ( \frac{AUC in ligand treated wells}{AUC of vehicle \text{-} treated control wells} ) ) .$$

Data are from a representative experiment.

### In Vivo Studies of PTHR1 Inverse Agonism

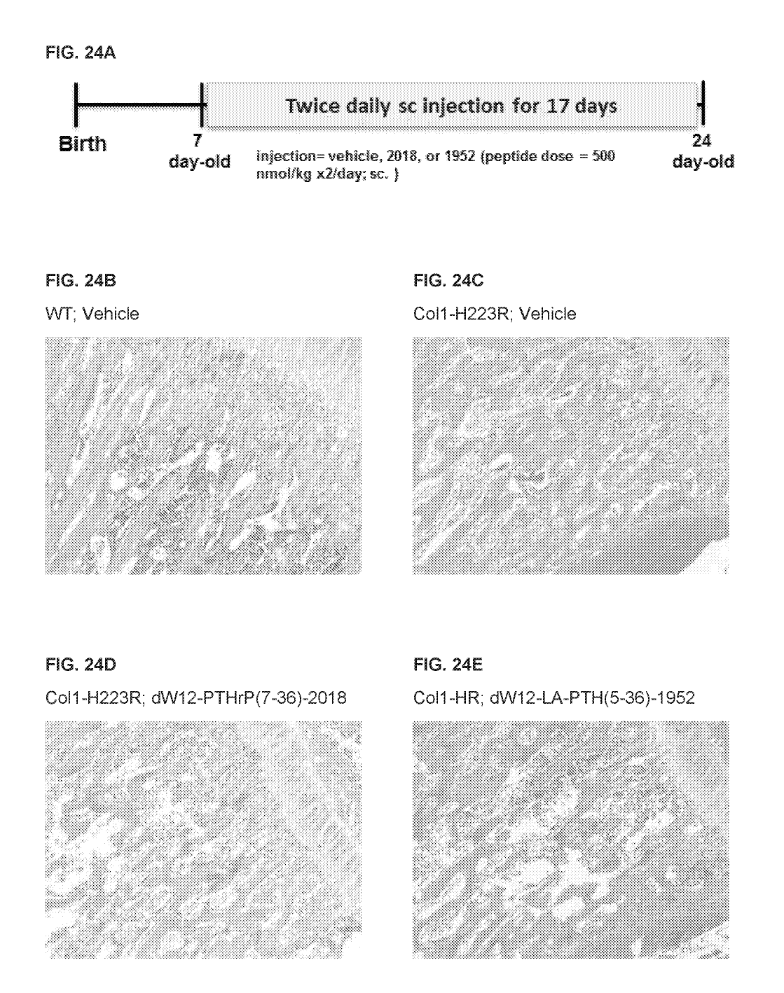

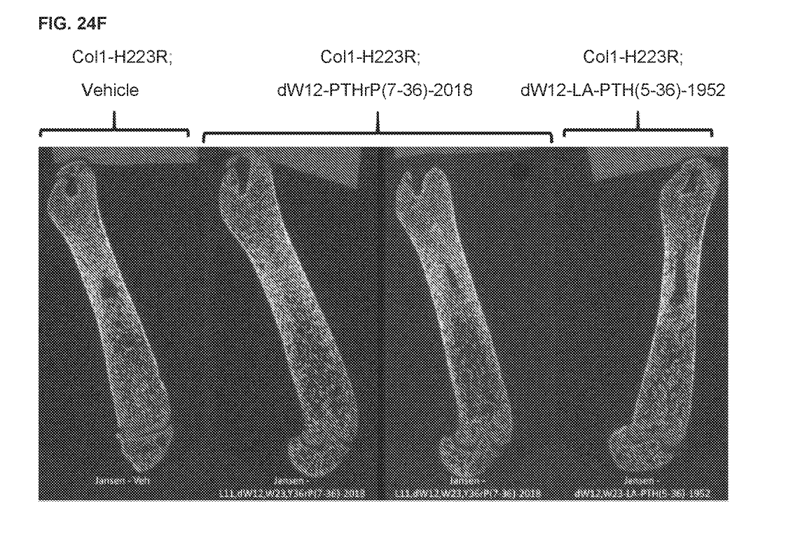

For the studies of inverse agonism at the PTHR1 in vivo, a transgenic mouse model of Jansen's disease, Col1-H223R, was used. In Col1-H223R mice, the human PTHR-H223R mutant allele is expressed specifically via the collagen-type-1a promoter in osteoblastic cells of bone. These mice are described by Calvi et al. (J. Clin. Invest., 107:277-286, 2001), and exhibit a high-bone mass phenotype. To assess the potential capacity of a new inverse agonist analog to correct the bone phenotype, Col1-H223R mice were injected subcutaneously twice daily for 17 days with dW12, W23-LA-PTH(5-36)-1952 (500 nmol/kg); in parallel, control population of Col1-H223R mice was receiving injections with vehicle or peptide L11, dW12, W23, Y36-PTHrP(7-36)-2018 (500 nmol/kg) (FIG. 24A).

Bone structural parameters were assessed by hematoxylin and eosin (H&E) stain-based histology of tibiae and by micro CT of femurs (FIGS. 24B-24E). Compared to vehicle treatment, treatment with either peptide reduced the total bone mass, and excessive interstitial fibrosis (seen in the H&E stained sections) which characterize the mutant phenotype. These reductions indicate a capacity of the peptides to suppress the elevated rates of bone accrual and cellular fibrosis that characterize the H223R mutant phenotype.

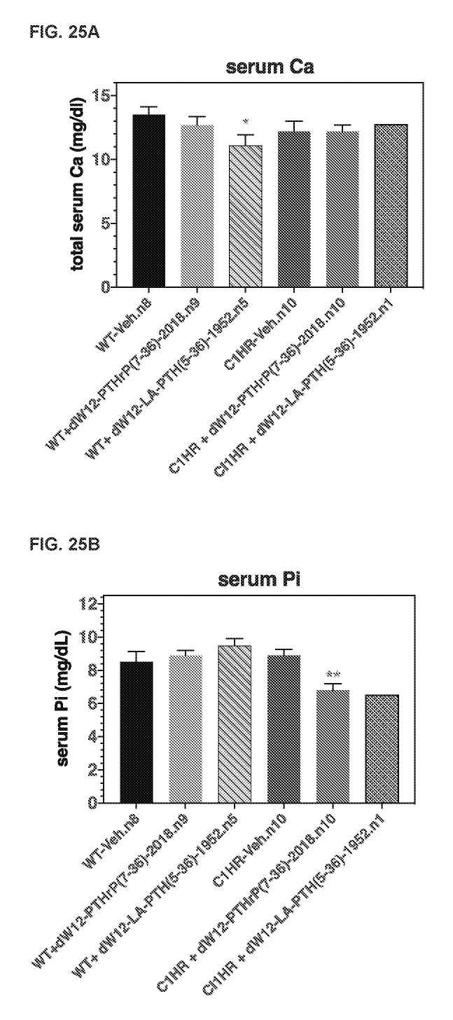

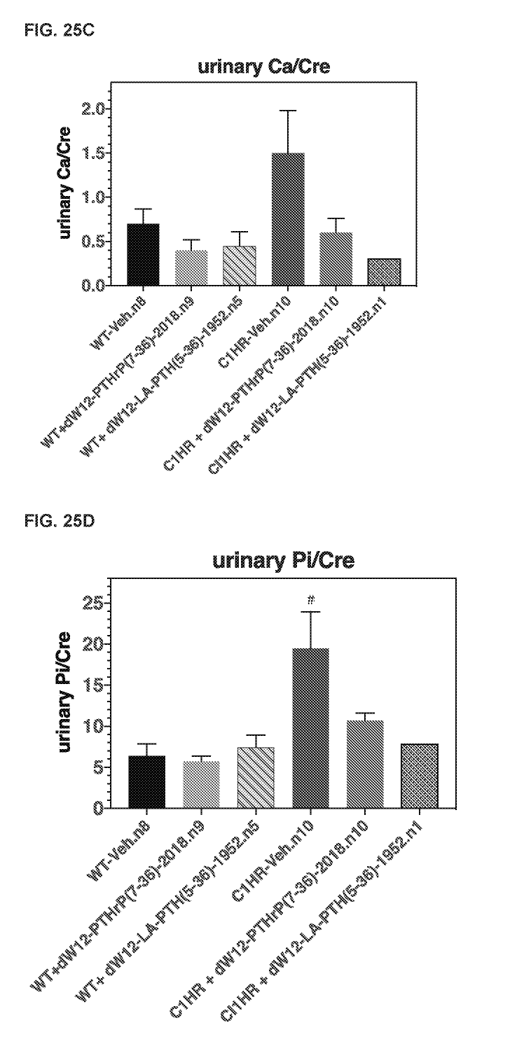

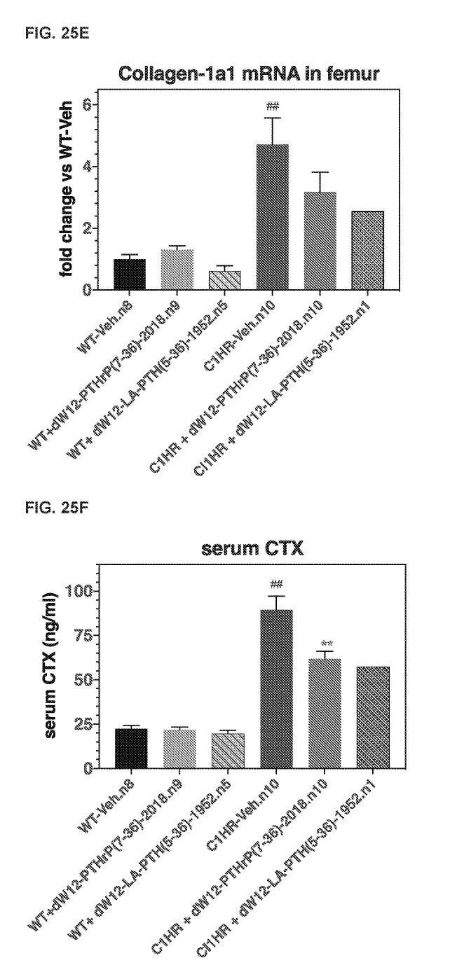

The effect of each regimen on markers of bone-turn-over (Ca, Pi, and CTX) in urine and/or blood and on gene mRNA expression in bone was measured. The results are illustrated in FIGS. 25A-25F.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A polypeptide or a pharmaceutically acceptable salt thereof comprising an N-terminally truncated PTH/PTHrP hybrid peptide or a fragment thereof, wherein said polypeptide is a PTHR1 antagonist or a PTHR1 inverse agonist.

2. The polypeptide of claim 1, wherein the N-terminally truncated PTH/PTHrP hybrid peptide is of formula (I):
(I)
Ile-Gln-Leu-X₀₁-His-X₀₂-X₀₃-X₀₄-X₀₅-X₀₆-X₀₇-X₀₈-X₀₉-
X₁₀-Arg-Arg-Arg-X₁₁-X₁₂-Leu-X₁₃-X₁₄-Leu-Ile-Ala-Glu-
Ile-His-Thr-Ala-Glu-X₁₅-Cys,
wherein
X₀₁ is Met, Leu, or Nle;
X₀₂ is Asn, Ala, Val, Asp, Glu, or Gln;
X₀₃ is Leu, Ala, Val, Met, Lys, Ile, Arg, Har, or Trp;
X₀₄ is Gly, Ala, His, Arg, or dTrp;
X₀₅ is Lys, Ala, Leu, Gln, Arg, His, or Trp;
X₀₆ is His, Leu, Arg, Phe, Trp, or Ala;
X₀₇ is Ile or Leu;
X₀₈ is Gln or Asn;
X₀₀ is Asp or Ser;
X₁₀ is Ala, Leu, Met, Glu, Ser, or Phe;
X₁₁ is Ala, Phe, Glu, Ser, Leu, Asn, Trp, or Lys;
X₁₂ is Phe or Trp;
X₁₃ is His, Arg, Leu, Trp, or Lys;
X₁₄ is Lys, His, Ala, Ser, Asn, or Arg; and
X₁₅ is Ile, Cys, or Tyr;
or a fragment thereof comprising from 24 to 32 contiguous amino acid residues of formula (I).

3. The polypeptide of claim 2, wherein the polypeptide is a fragment of the N-terminally truncated PTH/PTHrP hybrid peptide comprising amino acid residues 1-32 of formula (I).

4. The polypeptide of claim 2, wherein the polypeptide is a fragment of the N-terminally truncated PTH/PTHrP hybrid peptide comprising amino acid residues 3-32 of formula (I).

5. The polypeptide of claim 2, wherein the polypeptide is a fragment of the N-terminally truncated PTH/PTHrP hybrid peptide comprising amino acid residues 3-33 of formula (I).

6. The polypeptide of any one of claims 2 to 5, wherein X₀₁ is Met, X₀₄ is Ala, X₁₂ is Phe, and X₁₅ is Ile.

7. The polypeptide of any one of claims 2 to 5, wherein X₀₁ is Met, X₀₄ is dTrp, X₁₂ is Trp, and X₁₄ is Ile.

8. The polypeptide of any one of claims 2 to 5, wherein X₀₁ is Nle, X₀₄ is dTrp, X₁₂ is Trp, and X₁₅ is Tyr.

9. The polypeptide of any one of claims 2 to 5, wherein X₀₁ is Nle, X₀₄ is dTrp, X₁₂ is Trp, and X₁₅ is Cys.

10. The polypeptide of any one of claims 2 to 5, wherein X₀₁ is Nle, X₀₄ is dTrp, X₁₂ is Trp, and X₁₅ is Ile.

11. The polypeptide of any one of claims 2 to 5, wherein X₀₁ is Met, X₀₄ is dTrp, X₁₂ is Trp, and X₁₅ is Ile.

12. The polypeptide of claim 2, wherein the N-terminally truncated PTH/PTHrP hybrid peptide has the amino acid sequence
(SEQ ID NO: 1)
Ile-Gln-Leu-Met-His-Gln-Arg-Ala-Lys-Trp-Ile-Gln-
Asp-Ala-Arg-Arg-Arg-Ala-Phe-Leu-His-Lys-Leu-Ile-
Ala-Glu-Ile-His-Thr-Ala-Glu-Ile;
(SEQ ID NO: 2)
Ile-Gln-Leu-Met-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile;
(SEQ ID NO: 3)
Ile-Gln-Leu-Nle-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Tyr;
(SEQ ID NO: 4)
Ile-Gln-Leu-Nle-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Cys;
(SEQ ID NO: 5)
Ile-Gln-Leu-Nle-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile-Cys;
(SEQ ID NO: 6)
Ile-Gln-Leu-Met-His-Gln-Arg-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile-Cys;
(SEQ ID NO: 14)
Leu-Met-His-Gln-Arg-dTrp-Lys-Trp-Ile-Gln-Asp-Ala-
Arg-Arg-Arg-Ala-Trp-Leu-His-Lys-Leu-Ile-Ala-Glu-
Ile-His-Thr-Ala-Glu-Ile;
(SEQ ID NO: 15)
Leu-Nle-His-Gln-Arg-dTrp-Lys-Trp-Ile-Gln-Asp-Ala-
Arg-Arg-Arg-Ala-Trp-Leu-His-Lys-Leu-Ile-Ala-Glu-
Ile-His-Thr-Ala-Glu-Ile;
(SEQ ID NO: 16)
Leu-Nle-His-Gln-Leu-dTrp-Lys-Trp-Ile-Gln-Asp-Ala-
Arg-Arg-Arg-Ala-Trp-Leu-His-Lys-Leu-Ile-Ala-Glu-
Ile-His-Thr-Ala-Glu-Ile;
(SEQ ID NO: 17)
Ile-Gln-Leu-Nle-His-Gln-Leu-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile;
(SEQ ID NO: 18)
Leu-Met-His-Gln-Leu-dTrp-Lys-Trp-Ile-Gln-Asp-Ala-
Arg-Arg-Arg-Ala-Trp-Leu-His-Lys-Leu-Ile-Ala-Glu-
Ile-His-Thr-Ala-Glu-Ile;
or
(SEQ ID NO: 19)
Ile-Gln-Leu-Met-His-Gln-Leu-dTrp-Lys-Trp-Ile-Gln-
Asp-Ala-Arg-Arg-Arg-Ala-Trp-Leu-His-Lys-Leu-Ile-
Ala-Glu-Ile-His-Thr-Ala-Glu-Ile;
or a 3-32 or 3-33 fragment thereof.

13. The polypeptide of claim 1 or 2, further comprising a radionuclide, a polyethylene glycol, or a dye.

14. A pharmaceutical composition comprising the polypeptide of any one of claims 1 to 13 and a pharmaceutically acceptable carrier.

15. A method of antagonizing or inversely agonizing the activity of parathyroid hormone receptor 1 (PTHR1) in a cell, the method comprising contacting the cell with the polypeptide of any one of claims 1 to 13.

16. The method of claim 15, wherein the cell is a human cell.

17. A method of treating a disease or condition associated with PTHR1 signaling overactivity, the method comprising administering to the subject an effective amount of the polypeptide of any one of claims 1 to 13 or the pharmaceutical composition of claim 14.

18. The method of claim 17, wherein the disease or condition is hypercalcemia, hypophosphatemia, hyperparathyroidism, or Jansen's chondrodysplasia.

19. The method of claim 17 or 18, wherein the administering comprises subcutaneous, intravenous, intranasal, transpulmonary, transdermal, transmucosal, or oral administration of the polypeptide or the pharmaceutical composition to the subject.
